(19) United States
(12) Patent Application Publication
(10) Pub. No.: US 2025/0281048 A1
(43) Pub. Date: Sep. 11, 2025

(54) PREDICTION OF USER CHARACTERISTICS USING SENSOR SIGNALS

(71) Applicant: QUALCOMM Incorporated, San Diego, CA (US)

(72) Inventors: Rathin RADHAKRISHNAN NAIR, Tonawanda, NY (US); Fitzgerald JOHN ARCHIBALD, Richmond Hill (CA); Emily Kathryn BROOKS, Amherst, NY (US); Shounak Uday GORE, Hyderabad (IN); John Keith SCHNEIDER, Williamsville, NY (US)

(21) Appl. No.: 18/601,889

(22) Filed: Mar. 11, 2024

(51) Int. Cl. A61B 5/02; A61B 5/00; A61B 5/021; A61B 90/00; G16H 10/60; G16H 50/20; G16H 50/70

(52) U.S. Cl. CPC A61B 5/02007; A61B 5/0095; A61B 5/02125; A61B 5/742; A61B 90/06; G16H 10/60; G16H 50/20; G16H 50/70; A61B 5/681; A61B 2090/061; A61B 2090/064; A61B 2560/0462

(57) ABSTRACT

Methods and apparatus predicting a user characteristic using a category-based model are disclosed. In some embodiments, techniques may include: obtaining, by a control system, one or more measurements from a target object of a user using one or more sensors; determining, by the control system, at least one physiological characteristic associated with the user based on the one or more measurements from the user; predicting, by the control system, at least one secondary characteristic associated with the user, based on the at least one physiological characteristic associated with the user; and outputting, by the control system, the predicted at least one secondary characteristic associated with the user.

### TECHNICAL FIELD

This disclosure relates generally to devices and systems using sensors, including biometric sensors and signal data used in conjunction with a predictive methodology.

### DESCRIPTION OF RELATED TECHNOLOGY

Sensing technologies can be implemented in devices that can be used for various applications, including biometric sensing as but one example. Some such devices are, or include, photoacoustic sensors, which can address limitations in the usability of traditional measuring devices for continuous, noninvasive and/or ambulatory monitoring. Such devices and sensors can have a variety of uses, such as biomedical applications including health and wellness monitoring, or population studies related thereto. Although previously deployed devices can enable estimations of physiological parameters, there may be undiscovered yet useful user characteristics that are not directly measurable by these devices.

### SUMMARY

The systems, methods and devices of this disclosure each have several aspects, no single one of which is solely responsible for the desirable attributes disclosed herein.

In one aspect of the present disclosure, a method of predicting a user characteristic using a category-based model is disclosed. In some embodiments, the method may include: obtaining, by a control system, one or more measurements from a target object of a user using one or more sensors; determining, by the control system, at least one physiological characteristic associated with the user based on the one or more measurements from the user; predicting, by the control system, at least one secondary characteristic associated with the user, based on the at least one physiological characteristic associated with the user; and outputting, by the control system, the predicted at least one secondary characteristic associated with the user.

In another aspect of the present disclosure, an apparatus is disclosed. In some embodiments, the apparatus may include: one or more sensors; and a control system comprising one or more processors configured to: obtain one or more measurements from a target object of a user using the one or more sensors; determine at least one physiological characteristic associated with the user based on the one or more measurements from the user; predict at least one secondary characteristic associated with the user, based on the at least one physiological characteristic associated with the user; and output the predicted at least one secondary characteristic associated with the user.

In some embodiments, the apparatus may include: means for obtaining one or more measurements from a target object of a user using one or more sensors; means for determining at least one physiological characteristic associated with the user based on the one or more measurements from the user; means for predicting at least one secondary characteristic associated with the user, based on the at least one physiological characteristic associated with the user; and means for outputting the predicted at least one secondary characteristic associated with the user.

In another aspect of the present disclosure, a non-transitory computer-readable apparatus is disclosed. In some embodiments, the non-transitory computer-readable apparatus may include a storage medium, the storage medium comprising a plurality of instructions configured to, when executed by one or more processors of a control system, cause an apparatus to: obtain, by the control system, one or more measurements from a target object of a user using one or more sensors; determine, by the control system, at least one physiological characteristic associated with the user based on the one or more measurements from the user; predict, by the control system, at least one secondary characteristic associated with the user, based on the at least one physiological characteristic associated with the user; and output, by the control system, the predicted at least one secondary characteristic associated with the user.

Details of one or more implementations of the subject matter described in this disclosure are set forth in the accompanying drawings and the description below. Other features, aspects, and advantages will become apparent from the description, the drawings and the claims. Note that the relative dimensions of the following figures may not be drawn to scale.

### BRIEF DESCRIPTION OF THE DRAWINGS

FIG. 1 shows an example of a blood pressure monitoring device based on photoacoustic plethysmography, which may be referred to herein as PAPG.

FIG. 2 is a block diagram that shows example components of a sensor apparatus according to some disclosed implementations.

FIG. 3 shows examples of heart rate waveform (HRW) features that may be extracted according to some implementations.

FIG. 4A shows an example monitoring device designed to be worn around a wrist according to some implementations.

FIG. 4B shows an example monitoring device designed to be worn on a finger according to some implementations.

FIG. 4C shows an example monitoring device designed to reside on an earbud according to some implementations.

FIG. 5A-5C are block diagrams illustrating mechanisms for training a machine learning model, according to some approaches.

FIG. 6 shows an example generation and use of a single trained model.

FIG. 7 shows an example generation and use of multiple trained models, according to some embodiments.

FIG. 8 shows a more specific example clustering process for inference with multiple trained models is shown, according to some embodiments.

FIG. 9 shows a flow diagram of a method of obtaining one or more machine learning models configured to predict a user characteristic, according to some embodiments.

FIG. 10 shows a flow diagram of a method of predicting a user characteristic using a category-based model, according to some embodiments.

Like reference numbers and designations in the various drawings indicate like elements.

### DETAILED DESCRIPTION

The following description is directed to certain implementations for the purposes of describing various aspects of this disclosure. However, a person having ordinary skill in the art will readily recognize that the teachings herein can be applied in a multitude of different ways. Some of the concepts and examples provided in this disclosure are especially applicable to blood pressure monitoring applications or monitoring of other physiological parameters, characteristics, or properties of a target object of a user, such as a blood vessel. However, some implementations also may be applicable to other types of biological sensing applications, as well as to other fluid flow systems. The described implementations may be implemented in any device, apparatus, or system that includes an apparatus as disclosed herein. In addition, it is contemplated that the described implementations may be included in or associated with a variety of electronic devices such as, but not limited to: mobile telephones, multimedia Internet enabled cellular telephones, mobile television receivers, wireless devices, smartphones, smart cards, wearable devices such as bracelets, armbands, wristbands, rings, headbands, patches, chest bands, anklets, etc., Bluetooth® devices, personal data assistants (PDAs), wireless electronic mail receivers, hand-held or portable computers, netbooks, notebooks, smartbooks, tablets, printers, copiers, scanners, facsimile devices, global positioning system (GPS) receivers/navigators, cameras, digital media players, game consoles, wrist watches, clocks, calculators, television monitors, flat panel displays, electronic reading devices (e.g., e-readers), mobile health devices, computer monitors, auto displays (including odometer and speedometer displays, etc.), cockpit controls and/or displays, camera view displays (such as the display of a rear view camera in a vehicle), architectural structures, microwaves, refrigerators, stereo systems, cassette recorders or players, DVD players, CD players, VCRs, radios, portable memory chips, washers, dryers, washer/dryers, parking meters, automobile doors, Internet of Things (IoT) devices, etc. Thus, the teachings are not intended to be limited to the specific implementations depicted and described with reference to the drawings; rather, the teachings have wide applicability as will be readily apparent to persons having ordinary skill in the art.

As used herein, a “module” may refer to at least a portion of computer-executable instructions. In some embodiments, a module may be implemented by a hardware processor and/or a storage device configured to execute the corresponding computer-executable instructions. A hardware processor may include an integrated circuit device associated with a computing device, such as a server or a user device (e.g., a wearable device, a desktop computer, a laptop computer, a tablet computer, a mobile phone, or the like), which is programmable to perform specific tasks. In some embodiments, multiple modules may be implemented as a single module. In some embodiments, a single module may be implemented as multiple modules. In some embodiments, two or more modules may be executable by the same device (e.g., the same server, the same computing device).

Accurate, non-invasive, continuous monitoring devices for both clinical and consumer applications (e.g., for measuring physiological parameters such as blood pressure of a user) using a user-wearable device is made possible using sensors leveraging biometric signals such as photoacoustic signals. Such non-invasive and continuous monitoring devices could open avenues for efficient and effective diagnosis and treatment of cardiovascular conditions (e.g., hypertension), cardiovascular event detection, and stress monitoring. They would also allow daily spot checks of cardiovascular conditions including blood pressure, as well as overnight sleep monitoring.

However, in previously disclosed devices, such measurements may be limited to detecting known correlations between the measurements and the physiological parameters such as blood pressure. That is, the purpose of these monitoring devices may be limited to monitoring blood pressure or similar parameters and metrics. While this may be useful for determining anticipated conditions (e.g., high, medium, low blood pressure), there are many features within the biometric signals from which other types of insights may be determined according to devices and methods disclosed herein.

For example, in some approaches, features obtained from photoacoustic signals may indicate or enable estimation of physiological characteristics such as arterial stiffness, strain, stress, distension, compliance, dimension(s), pulse wave velocity (PWV), and/or other indication of deformation or force applied to a blood vessel of a user. Such physiological characteristics may be determined based on, for example, analyses of images obtained from the photoacoustic signals. A diameter of a blood vessel and/or an arterial cross-sectional area (which may be examples of dimensions) may be determined from the analysis of a photoacoustic image, for instance, and other characteristics may be estimated based thereon. Such characteristics may have a correlation to secondary characteristics associated with a user, such as the user's domicile or geographic location, behavioral pattern or habits (e.g., smoking), current behaviors, activity levels, other linked physiological parameters including biometric indications (e.g., blood glucose level, blood oxygen), and other user-related metrics including those that may not be expected. In one particular example, a high arterial strain indicative of stiff arteries may have a correlation with the user's smoking behaviors or habits, or with the user's living conditions in a polluted area. Such correlations may not be directly observable or detectable through the photoacoustic signals themselves. There may also be unexpected correlations between the photoacoustic signals and the secondary characteristics that are not obvious.

In some implementations, machine learning can be used to train machine learning (ML) or artificial intelligence models that can predict a secondary characteristic (e.g., associated with the user based on different features of physiological signals. In some implementations, multiple ML models may be used (in concert as an ensemble model in some cases), which may diversify the types of predictions based on multiple signal features. Unsupervised learning methodologies can be used to determine relationships or correlations between similar signal feature categories and secondary characteristics. Training user data may be categorized based on similar signal features (e.g., three categories such as stiffness, strain, stress) during training. Input user data during inference may be categorized similarly (e.g., automatically using a clustering algorithm, or manually) to generate a prediction of secondary characteristic(s). Signal data obtained using a device can thereby move beyond the limits of predefined categories and results, and may provide greater insights about a user or a group of users from he collected biometric data.

Particular aspects of the subject matter described in this disclosure can be implemented to realize one or more of the following potential advantages. According to implementations, by categorizing user data based on signal features and developing multiple ML models, modeling performance can be improved compared to using a single ML model. ML modeling can also allow for specialized, personalized, tailored modeling for groups of users. For instance, users may be grouped geographically or monitored for smoking patterns.

Additional details will follow after an initial description of relevant systems and technologies.

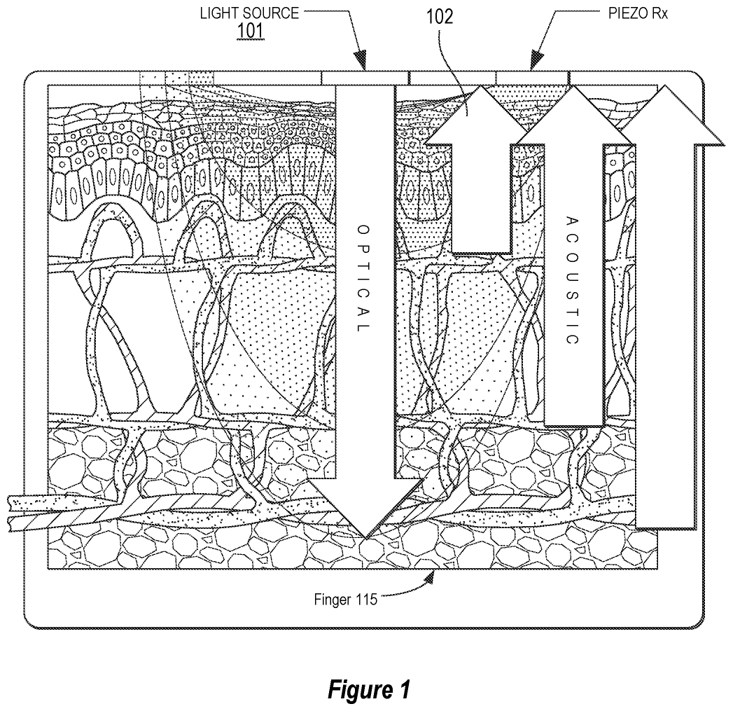

FIG. 1 shows an example of a blood pressure monitoring device based on photoacoustic plethysmography, which is referred to herein as PAPG. FIG. 1 shows the same examples of arteries, veins, arterioles, venules and capillaries inside a body part, which is a finger 115 in this example. In some examples, the light source 101 shown in FIG. 1 may be coupled to a light source system (not shown) that is disposed remotely from the body part (e.g., finger 115). In some implementations, the light source 101 may be an opening of an optical fiber or other waveguide. Such an opening may also be connected to an opening of an interface that is contactable with the body part. In some embodiments, the light source system may include one or more LEDs, one or more laser diodes, etc. In this example, the light source 101 has transmitted light (in some examples, green, red, infrared, and/or near-infrared (NIR) light) that has penetrated the tissues of the finger 115 in an illuminated zone.

In the example shown in FIG. 1, blood vessels (and components of the blood itself) are heated by the incident light from the light source 101 and are emitting acoustic waves 102. In this example, the emitted acoustic waves 102 include ultrasonic waves. According to this implementation, the acoustic wave emissions 102 are being detected by an ultrasonic receiver, which is a piezoelectric receiver in this example. Photoacoustic emissions 102 from the illuminated tissues, detected by the piezoelectric receiver, may be used to detect volumetric changes in the blood of the illuminated zone of the finger 115 that correspond to physiological data within the illuminated tissues of finger 115, such as heart rate waveforms. Although some of the tissue areas shown to be illuminated are offset from those shown to be producing photoacoustic emissions 102, this is merely for illustrative convenience. It will be appreciated that that the illuminated tissues will actually be those producing photoacoustic emissions. Moreover, it will be appreciated that the maximum levels of photoacoustic emissions will often be produced along the same axis as the maximum levels of illumination.

One important difference between an optical technique such as a photoplethysmography (PPG)-based system the PAPG-based method of FIG. 1 is that the acoustic waves shown in FIG. 1 travel much more slowly than the reflected light waves involved in PPG. Accordingly, depth discrimination based on the arrival times of the acoustic waves shown in FIG. 1 is possible, whereas depth discrimination based on the arrival times of the light waves in PPG may not be possible. This depth discrimination allows some disclosed implementations to isolate acoustic waves received from the different blood vessels.

According to some such examples, such depth discrimination allows artery heart rate waveforms to be distinguished from vein heart rate waveforms and other heart rate waveforms. Therefore, blood pressure estimation based on depth-discriminated PAPG methods can be substantially more accurate than blood pressure estimation based on PPG-based methods.

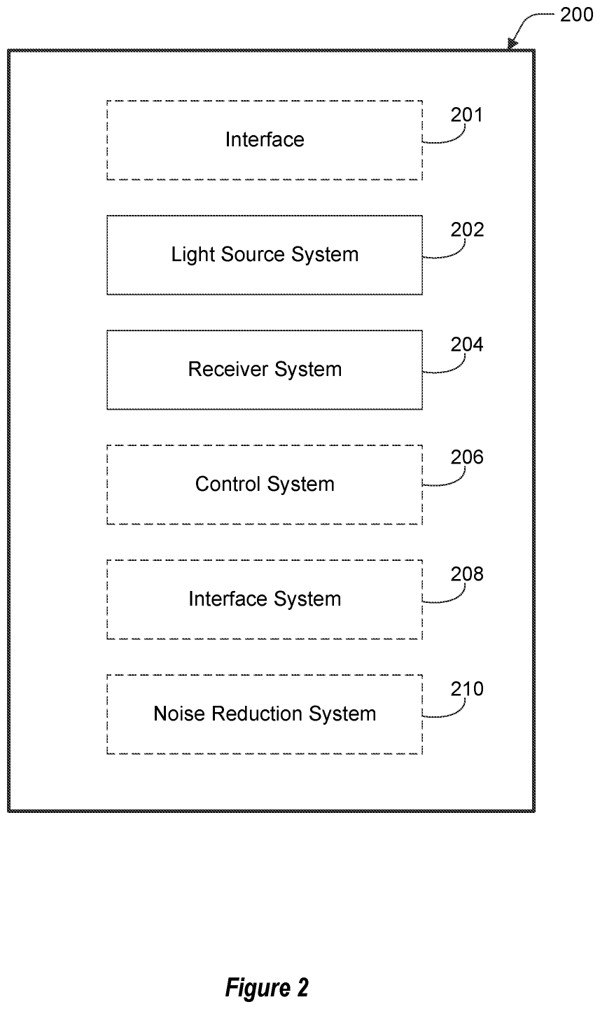

FIG. 2 is a block diagram that shows example components of a sensor apparatus 200 according to some implementations. In this example, the sensor apparatus 200 may include a light source system 202 and a receiver system 204. Some implementations of the sensor apparatus 200 may include an interface 201. Some implementations of the sensor apparatus 200 may include control system 206, an interface system 208, a noise reduction system 210, or a combination thereof.

In some embodiments, the light source system 202 and the receiver system 204 may be components of a photoacoustic (PAPG) sensor of the sensor apparatus 200. In various implementations described herein, the PAPG sensor and/or its components may operate in concert with physical components of the sensor apparatus 200 (e.g., cuff, band, ring, housing, coupling elements).

Various configurations of light source system 202 and receiver system 204 are disclosed herein. Specific examples are described in more detail below.

Some disclosed PAPG sensors described herein may include a platen, a light source system, and an ultrasonic receiver system. According to some implementations, the light source system may include a light source configured to produce and direct light. In some implementations, the platen may include an anti-reflective layer, a mirror layer, or combinations thereof. According to some implementations, the platen may have an outer surface, or a layer on the outer surface, with an acoustic impedance that is configured to approximate the acoustic impedance of human skin. In some implementations, the platen may have a surface proximate the ultrasonic receiver system, or a layer on the surface proximate the ultrasonic receiver system, with an acoustic impedance that is configured to approximate the acoustic impedance of the ultrasonic receiver system.

Some disclosed PAPG sensors described herein may include an interface, a light source system and an ultrasonic receiver system. Some such devices may not include a rigid platen. According to some implementations, the interface may be a physical, flexible interface constructed of one or more of suitable materials having a desired property or properties (e.g., an acoustic property such as acoustic impedance, softness of the material). In some implementations, the interface may be a flexible interface that can contact a target object that may be proximate to or contact the interface. There may be salient differences between such an interface and a platen. In some implementations, the light source system may be configured to direct light using one or more optical waveguides (e.g., optical fibers) configured to direct light toward a target object. According to some implementations, the interface may have an outer surface, or a layer on the outer surface, with an acoustic impedance that is configured to approximate the acoustic impedance of human skin. Such outer surface may have a contact portion that is contactable by a user or a body part of the user (e.g., finger, wrist). In some examples, the optical waveguide(s) may be embedded in one or more acoustic matching layers that are configured to bring the light transmitted by the optical waveguide(s) very close to tissue. The outer surface and/or other parts of the interface may be compliant, pliable, flexible, or otherwise at least partially conforming to the shape and contours of the body part of the user. In some implementations, the interface may have a surface proximate the ultrasonic receiver system, or a layer on the surface proximate the ultrasonic receiver system, with an acoustic impedance that is configured to approximate the acoustic impedance of the ultrasonic receiver system.

In implementations of the sensor apparatus 200 that include an interface 201, the platen or the interface may be an example of the interface 201. In some implementations in which the receiver system 204 includes an ultrasonic receiver system, the interface 201 may be an interface having a contact portion configured to make contact with a body part of a user such as the finger 115 shown in FIG. 1.

In some embodiments, the light source system 202 may be an example of a light source system that is coupled to a light source 101 as shown in FIG. 1. That is to say, in some implementations, the light source system 202 may be configured to generate optical signals and trigger, generate, create, or otherwise cause a photoacoustic (PAPG) response from a target object, such as a blood vessel or other tissue, and cause emission of acoustic waves (e.g., acoustic waves 102 as illustrated in FIG. 1).

According to some embodiments, the light source system 202 may include one or more light sources configured to produce and direct light. In some implementations, the light source system 202 may include one or more light-emitting diodes (LEDs). In some implementations, the light source system 202 may include one or more laser diodes. According to some implementations, the light source system 202 may include one or more vertical-cavity surface-emitting lasers (VCSELs). In some implementations, the light source system 202 may include one or more edge-emitting lasers. In some implementations, the light source system 202 may include one or more neodymium-doped yttrium aluminum garnet (Nd:YAG) lasers. In some implementations, the light source system 202 may include at least one multi-junction laser diode, which may produce less noise than single-junction laser diodes.

Hence, the light source system 202 may include, for example, a laser diode, a light-emitting diode (LED), or a line or an array of either or both. In addition, in some implementations, the light source (e.g., laser diode, LED) of the light source system 202 may be steered to different directions and locations. A line or an array of light sources of the light source system 202 can similarly be steered individually or collectively. The light source system 202 may be configured to, via the laser diode(s) or LEDs, generate and emit optical signals. The light source system 202 may, in some examples, be configured to transmit light or optical signals in one or more wavelength ranges. An example wavelength range for the light source system 202 may be 400 to 1200 nanometers (nm). Some applications may use higher wavelengths, as noted below. In some examples, the light source system 202 may be configured to transmit light in a wavelength range of 500 to 600 nm. According to some examples, the light source system 202 may be configured to transmit light in a wavelength range of 800 to 950 nm. According to some examples, the light source system 202 may be configured to transmit light in infrared or near infrared (NIR) region of the electromagnetic spectrum (about 700 to 2500 nm). In view of factors such as skin reflectance, fluence, the absorption coefficients of blood and various tissues, and skin safety limits, one or both of these wavelength ranges may be suitable for various use cases. For example, the wavelength ranges of 500 nm to 600 nm and of 800 to 950 nm may both be suitable for obtaining photoacoustic responses from relatively smaller, shallower blood vessels, such as blood vessels having diameters of approximately 0.5 mm and depths in the range of 0.5 mm to 1.5 mm, such as may be found in a finger. The wavelength range of 800 to 950 nm, or about 700 to 900 nm, or about 600 to 1100 nm may, for example, be suitable for obtaining photoacoustic responses from relatively larger, deeper blood vessels, such as blood vessels having diameters of approximately 2.0 mm and depths in the range of 2 mm to 3 mm, such as may be found in an adult wrist. In some implementations, the light source system 202 may be configured to switch wavelengths to capture acoustic information from different depths, e.g., based on signal(s) from the control system 206.

In some implementations, the light source system 202 may be configured for emitting one or more wavelengths of light, which may be selectable to trigger acoustic wave emissions primarily from a particular type of material. For example, because the hemoglobin in blood absorbs near-infrared light very strongly, in some implementations, the light source system 202 may be configured for emitting one or more wavelengths of light in the near-infrared range, in order to trigger acoustic wave emissions from hemoglobin. However, in some examples, the control system 206 may control the wavelength(s) of light emitted by the light source system 202 to preferentially induce acoustic waves in blood vessels, other soft tissue, and/or bones. For example, an infrared (IR) light-emitting diode LED may be selected and a short pulse of IR light emitted to illuminate a portion of a target object and generate acoustic wave emissions that are then detected by the receiver system 204. In some implementations, the light source system 202 may be configured to select specific wavelength values, such as 808 nm, 905 nm, or 940 nm. In another example, an IR LED and a red LED or other color such as green, blue, white or ultraviolet (UV) may be selected and a short pulse of light emitted from each light source in turn with ultrasonic images obtained after light has been emitted from each light source. In other implementations, one or more light sources of different wavelengths may be fired in turn or simultaneously to generate acoustic emissions that may be detected by an ultrasonic receiver of the receiver system 204. Image data from the ultrasonic receiver that is obtained with light sources of different wavelengths and at different depths (e.g., varying range gate delays (RGDs)) into the target object may be combined to determine the location and type of material in the target object. Image contrast may occur as materials in the body generally absorb light at different wavelengths differently. As materials in the body absorb light at a specific wavelength, they may heat differentially and generate acoustic wave emissions with sufficiently short pulses of light having sufficient intensities. Depth contrast may be obtained with light of different wavelengths and/or intensities at each selected wavelength. That is, successive images may be obtained at a fixed RGD (which may correspond with a fixed depth into the target object) with varying light intensities and wavelengths to detect materials and their locations within a target object. For example, hemoglobin, blood glucose or blood oxygen within a blood vessel inside a target object such as a finger may be detected photoacoustically.

In various implementations, the light source system 202 may be configured to emit pulses of light or optical signals having a pulse width. According to some implementations, the light source system 202 may be configured for emitting a light pulse with a pulse width less than about 100 nanoseconds. In some implementations, the light pulse may have a pulse width between about 10 nanoseconds and about 500 nanoseconds or more (e.g., from 3 nanoseconds to 1000 nanoseconds). In some cases, the pulse width may be selected from a range between about 50 nanoseconds and about 200 nanoseconds. According to some examples, the light source system 202 may be configured for emitting a plurality of light pulses at a pulse repetition frequency between 10 Hz and 100 kHz, or in some cases, between 50 Hz and 25 kHz, or between 1 kHz and 5 kHz. Alternatively, or additionally, in some implementations the light source system 202 may be configured for emitting a plurality of light pulses at a pulse repetition frequency between about 1 MHz and about 100 MHz. Alternatively, or additionally, in some implementations, the light source system 202 may be configured for emitting a plurality of light pulses at a pulse repetition frequency between about 10 Hz and about 1 MHz. In some examples, the pulse repetition frequency of the light pulses may correspond to an acoustic resonant frequency of the ultrasonic receiver and/or other parts of the sensor apparatus 200. For example, a set of four or more light pulses may be emitted from the light source system 202 at a frequency that corresponds with the resonant frequency of a resonant acoustic cavity in the sensor stack, allowing a build-up of the received ultrasonic waves and a higher resultant signal strength. In some implementations, filtered light or light sources with specific wavelengths for detecting selected materials may be included with the light source system 202. In some implementations, the light source system 202 may contain light sources such as red, green and blue LEDs of a display that may be augmented with light sources of other wavelengths (such as IR and/or UV) and with light sources of higher optical power. For example, high-power laser diodes or electronic flash units (e.g., an LED or xenon flash unit) with or without filters may be used for short-term illumination of the target object.

According to some examples, the light source system 202 may also include one or more light-directing elements configured to direct light from the light source system 202 towards the target object. In some examples, the one or more light-directing elements may include at least one diffraction grating. Alternatively, or additionally, the one or more light-directing elements may include at least one lens. In some implementations, the light source system 202 may be configured to direct light using one or more optical waveguides (e.g., optical fibers) configured to direct light toward the target object.

In various configurations, the light source system 202 may incorporate anti-reflection (AR) coating, a mirror, a light-blocking layer, a shield to minimize crosstalk, etc.

The light source system 202 may include various types of drive circuitry, depending on the particular implementation. In some examples, the light source system 202 may include a drive circuit (also referred to herein as drive circuitry) configured to cause the light source system 202 to emit pulses of light at pulse widths in a range from 3 nanoseconds to 1000 nanoseconds. According to some examples, the light source system 202 may include a drive circuit configured to cause the light source system 204 to emit pulses of light at pulse repetition frequencies in a range from 1 kilohertz to 100 kilohertz.

In some example implementations, some or all of the one or more light sources of the light source system 202 may be disposed at or along an axis that is parallel to or angled relative to a central axis associated with the sensor apparatus 200. Optical signals may be emitted toward a target object (e.g., blood vessel), which may cause generation of ultrasonic waves by the target object. These ultrasonic waves may be detectable by one or more receiver elements of a receiver system 204.

In some configurations, at least one additional light source system (having some or all components and functionalities described above) and/or at least one additional receiver system (having some or all components and functionalities described above) may be included with or associated with (e.g., communicatively coupled to) the sensor apparatus 200.

In some examples, the control system 206 may control the wavelength(s) of light emitted by the second light source system 203. The second light source system 203 may, in some examples, be configured to transmit light in a wavelength range of about 500 nm to 16000 nm (16 micrometers (μm)). In some examples, the second light source system 203 may be configured to transmit light in a wavelength range of 500 nm to 700 nm. In some examples, the second light source system 203 may be configured to transmit light in a wavelength of 1550 nm.

Various examples of a receiver system 204 are disclosed herein, some of which may include ultrasonic receiver systems, optical receiver systems, or combinations thereof. In some implementations, the receiver system 204 includes an ultrasonic receiver system having the one or more receiver elements. One or more of the receiver elements may include one or more photodetectors or photosensors. In implementations that include an ultrasonic receiver system, the ultrasonic receiver and an ultrasonic transmitter may be combined in an ultrasonic transceiver. In some examples, the receiver system 204 may include a piezoelectric receiver layer, such as a layer of PVDF polymer or a layer of PVDF-TrFE copolymer. In some implementations, a single piezoelectric layer may serve as an ultrasonic receiver. In some implementations, other piezoelectric materials may be used in the piezoelectric layer, such as aluminum nitride (AlN) or lead zirconate titanate (PZT). The receiver system 204 may, in some examples, include an array of ultrasonic transducer elements, such as an array of piezoelectric micromachined ultrasonic transducers (PMUTs), an array of capacitive micromachined ultrasonic transducers (CMUTs), etc. In some such examples, a piezoelectric receiver layer, PMUT elements in a single-layer array of PMUTs, or CMUT elements in a single-layer array of CMUTs, may be used as ultrasonic transmitters as well as ultrasonic receivers. According to some examples, the receiver system 204 may be, or may include, an ultrasonic receiver array. In some examples, the sensor apparatus 200 may include one or more separate ultrasonic transmitter elements or one or more separate arrays of ultrasonic transmitter elements. In some examples, the ultrasonic transmitter(s) may include an ultrasonic plane-wave generator.

In some implementations, at least portions of the sensor apparatus 200 (for example, the light source system 202, the receiver system 204, or a combination thereof) may include one or more sound-absorbing layers, acoustic isolation material, light-absorbing material, light-reflecting material, or combinations thereof. In some examples, acoustic isolation material may reside between the light source system 202 and at least a portion of the receiver system 204. In some examples, at least portions of the sensor apparatus 200 (for example, the light source system 202, the receiver system 204, or a combination thereof) may include one or more electromagnetically shielded transmission wires. In some such examples, the one or more electromagnetically shielded transmission wires may be configured to reduce electromagnetic interference from the light source system 202 that is received by the receiver system 204.

The control system 206 may include one or more general purpose single- or multi-chip processors, digital signal processors (DSPs), application specific integrated circuits (ASICs), field programmable gate arrays (FPGAs) or other programmable logic devices, discrete gates or transistor logic, discrete hardware components, or combinations thereof. The control system 206 also may include (and/or be configured for communication with) one or more memory devices, such as one or more random access memory (RAM) devices, read-only memory (ROM) devices, etc. Accordingly, the sensor apparatus 200 may have a memory system that includes one or more memory devices, though the memory system is not shown in FIG. 2. The control system 206 may be configured for receiving and processing data from the receiver system 204, e.g., as described below. If the sensor apparatus 200 includes an ultrasonic transmitter, the control system 206 may be configured for controlling the ultrasonic transmitter. In some implementations, functionality of the control system 206 may be partitioned between one or more controllers or processors, such as a dedicated sensor controller and an applications processor of a mobile device. The control system 206 may be configured to perform or cause performance of certain operations using one or more of the above components. Example configurations and operations will be described in greater detail.

In some examples, the control system 206 may be communicatively coupled to the light source system 202, and configured to control the light source system 202 to emit light towards a target object. In some such examples, the control system 206 may be configured to receive signals from the receiver system 204 (including one or more receiver elements) corresponding to the ultrasonic waves generated by the target object responsive to the light from the light source system. In some examples, the control system 206 may be configured to identify one or more blood vessel signals, such as arterial signals or vein signals, from the ultrasonic receiver system. In some such examples, the one or more arterial signals or vein signals may be, or may include, one or more blood vessel wall signals corresponding to ultrasonic waves generated by one or more arterial walls or vein walls of the target object. In some such examples, the one or more arterial signals or vein signals may be, or may include, one or more arterial blood signals corresponding to ultrasonic waves generated by blood within an artery of the target object or one or more vein blood signals corresponding to ultrasonic waves generated by blood within a vein of the target object. In some examples, the control system 206 may be configured to determine or estimate one or more physiological parameters or cardiac features based, at least in part, on one or more arterial signals, on one or more vein signals, or on combinations thereof. According to some examples, a physiological parameter may be, or may include, blood pressure. In some approaches, blood pressure can be estimated based at least on PWV, as will be discussed below.

In further examples, the control system 206 may be communicatively coupled to the receiver system 204. The receiver system 204 may be configured to detect acoustic signals from the target object. The control system 206 may be configured to select at least one of a plurality of receiver elements of the receiver system 204. Such selected receiver element(s) may correspond to the best signals from multiple receiver elements. In some embodiments, the selection of the at least one receiver element may be based on information regarding detected acoustic signals (e.g., arterial signals or vein signals) from the plurality of receivers. For example, signal quality or signal strength (based, e.g., on signal-to-noise ratio (SNR)) of some signals may be relatively higher than some others or above a prescribed threshold or percentile, which may indicate the best signals. In some implementations, the control system 206 may also be configured to, based on the information regarding detected acoustic signals, determine or estimate at least one characteristic of the blood vessels such as PWV (indicative of arterial stiffness), arterial dimensions, or both.

Some implementations of the sensor apparatus 200 may include an interface system 208. In some examples, the interface system 208 may include a wireless interface system. In some implementations, the interface system 208 may include a user interface system, one or more network interfaces, one or more interfaces between the control system 206 and a memory system and/or one or more interfaces between the control system 206 and one or more external device interfaces (e.g., ports or applications processors), or combinations thereof. According to some examples in which the interface system 208 is present and includes a user interface system, the user interface system may include a microphone system, a loudspeaker system, a haptic feedback system, a voice command system, one or more displays, or combinations thereof. According to some examples, the interface system 208 may include a touch sensor system, a gesture sensor system, or a combination thereof. The touch sensor system (if present) may be, or may include, a resistive touch sensor system, a surface capacitive touch sensor system, a projected capacitive touch sensor system, a surface acoustic wave touch sensor system, an infrared touch sensor system, any other suitable type of touch sensor system, or combinations thereof.

In some examples, the interface system 208 may include a force sensor system. The force sensor system (if present) may be, or may include, a piezo-resistive sensor, a capacitive sensor, a thin film sensor (for example, a polymer-based thin film sensor), another type of suitable force sensor, or combinations thereof. If the force sensor system includes a piezo-resistive sensor, the piezo-resistive sensor may include silicon, metal, polysilicon, glass, or combinations thereof. An ultrasonic fingerprint sensor and a force sensor system may, in some implementations, be mechanically coupled. In some implementations, the force sensor system may be mechanically coupled to a platen. In some such examples, the force sensor system may be integrated into circuitry of the ultrasonic fingerprint sensor. In some examples, the interface system 208 may include an optical sensor system, one or more cameras, or a combination thereof.

According to some examples, the sensor apparatus 200 may include a noise reduction system 210. For example, the noise reduction system 210 may include one or more mirrors that are configured to reflect light from the light source system 202 away from the receiver system 204. In some implementations, the noise reduction system 210 may include one or more sound-absorbing layers, acoustic isolation material, light-absorbing material, light-reflecting material, or combinations thereof. In some examples, the noise reduction system 210 may include acoustic isolation material, which may reside between the light source system 202 and at least a portion of the receiver system 204, on at least a portion of the receiver system 204, or combinations thereof. In some examples, the noise reduction system 210 may include one or more electromagnetically shielded transmission wires. In some such examples, the one or more electromagnetically shielded transmission wires may be configured to reduce electromagnetic interference from circuitry of the light source system, receiver system circuitry, or combinations thereof, that is received by the receiver system.

In some configurations, the light source system 202 and receiver system 204 may be implemented in the device such that, advantageously, no physical contact with the skin is needed (or even possible in normal operation), nor a coupling medium (e.g., a gel) between these components and the skin. As such, an interface 201 (e.g., a contact surface, flexible surface, or a platen) may not be present in the sensor apparatus 200, although other wearable or stabilizing structure may be present to secure the sensor apparatus 200 to the user.

In various embodiments described herein, the light source system 202 and the receiver system 204, or at least portions of their components, may be implemented with a device, such as a wearable device. In some embodiments, the sensor apparatus 200 may be a wearable device configured to be worn by a user, e.g., around the wrist, finger, arm, leg, ankle, waist, ear, neck, or another appendage, or another portion of the body. In an example implementation, the sensor apparatus 200 may have the form of a wristwatch and can be worn around the wrist. However, the embodiments described herein are not so limited. In certain cases, the components of the sensor apparatus 200 may not all be worn. For instance, a portion of the sensor apparatus 200 (e.g., the light source system 202 and/or the receiver system 204) may be worn around an appendage, but other components (e.g., the light source system 202 and/or the receiver system 204) may be in a separate sensor component and/or not be in a wearable chassis.

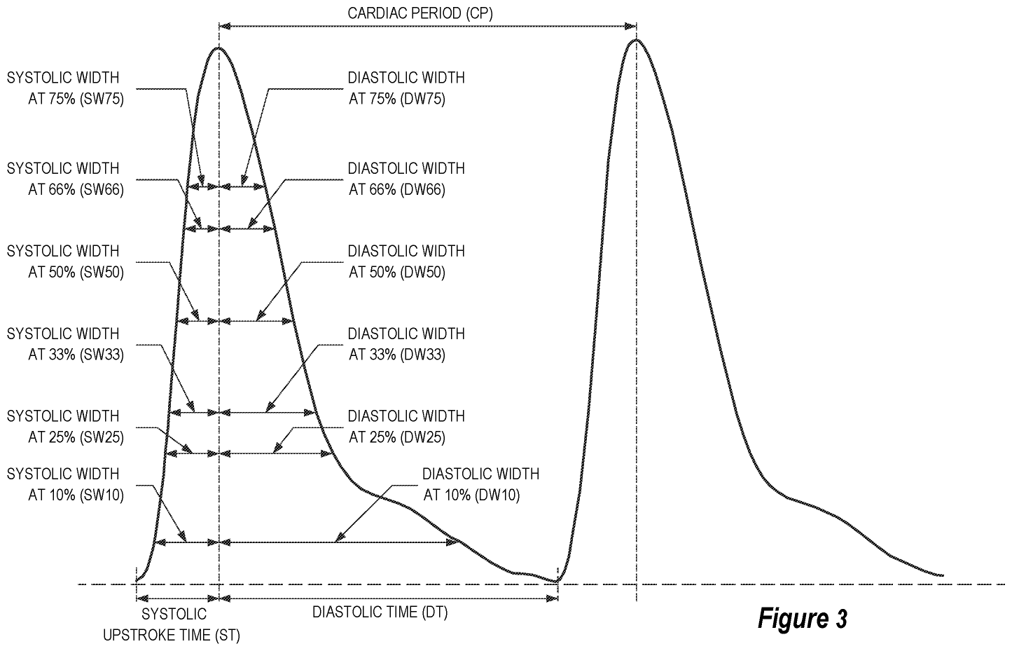

FIG. 3 shows examples of heart rate waveform (HRW) features that may be extracted according to some implementations. The horizontal axis of FIG. 3 represents time and the vertical axis represents signal amplitude. The cardiac period is indicated by the time between adjacent peaks of the HRW. The systolic and diastolic time intervals are indicated below the horizontal axis. During the systolic phase of the cardiac cycle, as a pulse propagates through a particular location along an artery, the arterial walls expand according to the pulse waveform and the elastic properties of the arterial walls. Along with the expansion is a corresponding increase in the volume of blood at the particular location or region, and with the increase in volume of blood an associated change in one or more characteristics in the region. Conversely, during the diastolic phase of the cardiac cycle, the blood pressure in the arteries decreases and the arterial walls contract. Along with the contraction is a corresponding decrease in the volume of blood at the particular location, and with the decrease in volume of blood an associated change in the one or more characteristics in the region.

The HRW features that are illustrated in FIG. 3 pertain to the width of the systolic and/or diastolic portions of the HRW curve at various “heights,” which are indicated by a percentage of the maximum amplitude. For example, the SW50 feature is the width of the systolic portion of the HRW curve at a “height” of 50% of the maximum amplitude. In some implementations, the HRW features used for blood pressure estimation may include some or all of the SW10, SW25, SW33, SW50, SW66, SW75, DW10, DW25, DW33, DW50, DW66 and DW75 HRW features. In other implementations, additional HRW features may be used for blood pressure estimation. Such additional HRW features may, in some instances, include the sum and ratio of the SW and DW at one or more “heights,” e.g., (DW75+SW75), DW75/SW75, (DW66+SW66), DW66/SW66, (DW50+SW50), DW50/SW50, (DW33+SW33), DW33/SW33, (DW25+SW25), DW25/SW25 and/or (DW10+SW10), DW10/SW10. Other implementations may use yet other HRW features for blood pressure estimation. Such additional HRW features may, in some instances, include sums, differences, ratios and/or other operations based on more than one “height,” such as (DW75+SW75)/(DW50+SW50), (DW50+SW50/(DW10+SW10), etc.

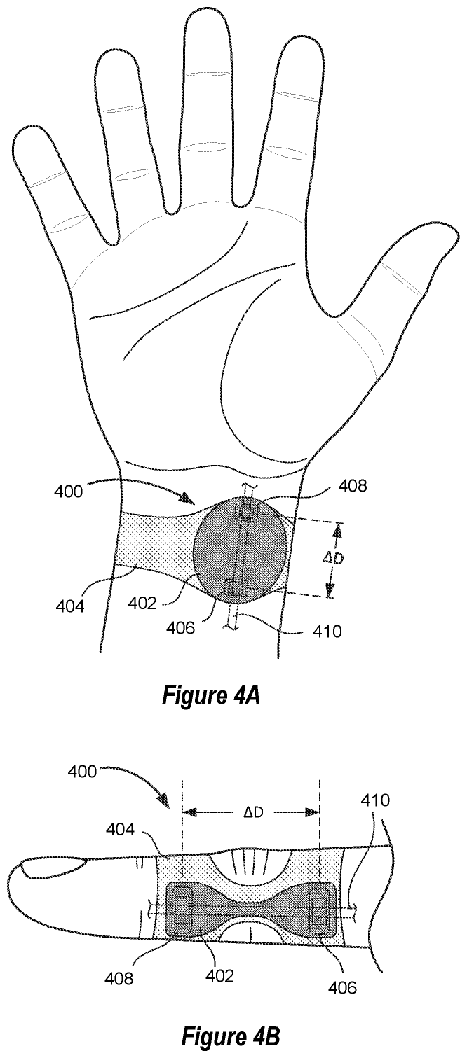

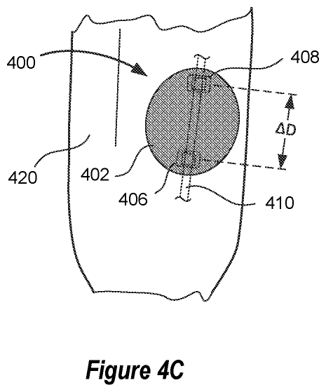

In some implementations, the monitoring device can be positioned around a wrist of a user with a strap or band, similar to a watch or fitness/activity tracker. FIG. 4A shows an example device 400 designed to be worn around a wrist according to some implementations. In some embodiments, the example device 400 may include the sensor apparatus 200 so as to allow components of the sensor apparatus 200 to interact with the user, e.g., via the skin of the user. In the illustrated example, the monitoring device 400 includes a housing 402 integrally formed with, coupled with or otherwise integrated with a wristband 404. The first and the second arterial sensors 406 and 408 may, in some instances, each include an instance of the ultrasonic receiver system and a portion of the light source system that are described above. In this example, the example device 400 is coupled around the wrist such that the first and the second arterial sensors 406 and 408 within the housing 402 are each positioned along a segment of the radial artery 410 (note that the sensors are generally hidden from view from the external or outer surface of the housing facing the subject while the monitoring device is coupled with the subject, but exposed on an inner surface of the housing to enable the sensors to obtain measurements through the subject's skin from the underlying artery). Also as shown, the first and the second arterial sensors 406 and 408 are separated by a fixed distance AD. In some other implementations, the example device 400 can similarly be designed or adapted for positioning around a forearm, an upper arm, an ankle, a lower leg, an upper leg, or a finger (all of which are hereinafter referred to as “limbs”) using a strap or band.

FIG. 4B shows an example device 400 designed to be worn on a finger according to some implementations. The first and the second arterial sensors 406 and 408 may, in some instances, each include an instance of the ultrasonic receiver and a portion of the light source system that are described above.

In some other implementations, the devices disclosed herein can be positioned on a region of interest of the user without the use of a strap or band. For example, the first and the second arterial sensors 406 and 408 and other components of the monitoring device can be enclosed in a housing that is secured to the skin of a region of interest of the user using an adhesive or other suitable attachment mechanism (an example of a “patch” monitoring device).

FIG. 4C shows an example device 400 designed to reside on an earbud according to some implementations. According to this example, the monitoring device 400 is coupled to the housing of an earbud 420. The first and second arterial sensors 406 and 408 may, in some instances, each include an instance of the ultrasonic receiver and a portion of the light source system that are described above.

### Example Applications of Machine Learning on Sensor Data

In traditional approaches to studying a group or population of users, users may be categorized into predefined or preset categories based on data collected from the users. For example, biometric data may be obtained using sensors capable of detecting physiological characteristics and changes in the users. Based on known or derived metrics, users may then be categorized into discrete categories, such as high, medium or low blood pressure for instance.

However, this limits the users into only those predefined categories. While such an approach may be useful in certain situations, and may simplify user categorization, much information about the users may remain unknown or undiscovered. It may be possible and desirable to obtain (or attempt to) greater insights about a user or a group of users from the collected data (e.g., biometric data). In fact, insights about users that may not even have been anticipated or expected may be discovered using methods, including automated methods such as machine learning, that study trends within a set of data.

As an illustrative example, high strain of a user's blood vessel (determined using a sensor, e.g., sensor apparatus 200) could mean that the user has stiff arteries. In some example cases, the high strain and/or stiff arteries may have been caused by smoking or smoking patterns of the user, or such smoking behavior may have been a primary or partial contributor to the high strain and/or stiff arteries. In some example cases, the high strain and/or stiff arteries may have been caused by living conditions in a polluted location or region (which may be defined by, e.g., zone or boundaries known to a system such as a network or a location server; geodetic or geographic coordinates; city, county, country, etc. with an air quality index (AQI) or other environmental metric that is tracked). In other cases, there may be different and possibly unknown relationships between (i) a physiological characteristic of a target object (e.g., stiffness, strain, stress, distension, compliance, dimension (e.g., diameter, cross-sectional area), PWV, other indication of deformation or force applied to a blood vessel of a user) or a physiological parameter of the user (e.g., blood pressure), and (ii) one or more secondary characteristics associated with the user. A secondary characteristic associated with a user may include, e.g., the user's domicile or geographic location, behavioral pattern or habits (e.g., smoking), current behaviors, activity levels, other linked physiological parameters including biometric indications (e.g., blood glucose level, blood oxygen), and other user-related metrics including those that may not be expected. A further example may include a metric associated with above, for example, a metric associated with the location of the user, e.g., AQI, average temperature, altitude, etc. which may affect the user's physiology or behaviors. A further example may be a mental, emotional, or psychological state of the user. which in some approaches may be based on location and biometric indications, where, for instance, a user at an office location having high blood pressure or high heart rate could indicate mental stress.

To reveal such trends or relationships, including those unexpected, unknown, or not obvious, between a user's physiology and a secondary characteristic, one or more models may be developed. In some embodiments, machine learning (ML) models may be trained and generated. In some approaches, users may be identified and categorized based on physiological features. For example, in a training dataset of users whose physiological characteristics have been measured, user data may be categorized into groups. As but one example, users may be grouped into those having a stress above a threshold, a strain above a threshold, and a stiffness above a threshold. Myriad categories may be possible, e.g., based on various physiological characteristics or parameters such as the above examples. Different training datasets can result in specialized, personalized, tailored models for various applications. These categories may relate to physiology-based features that may be extracted or obtained from sensor signals, e.g., PAPG signals obtained using a photoacoustic sensor of sensor apparatus 200. Features may thus be clustered and isolated to evaluate behavior and other patterns in the data. For example, a user's geographic location or smoking patterns may be identified based on stress, strain, or other physiological characteristics or parameters calculated from PAPG signal data as discussed above.

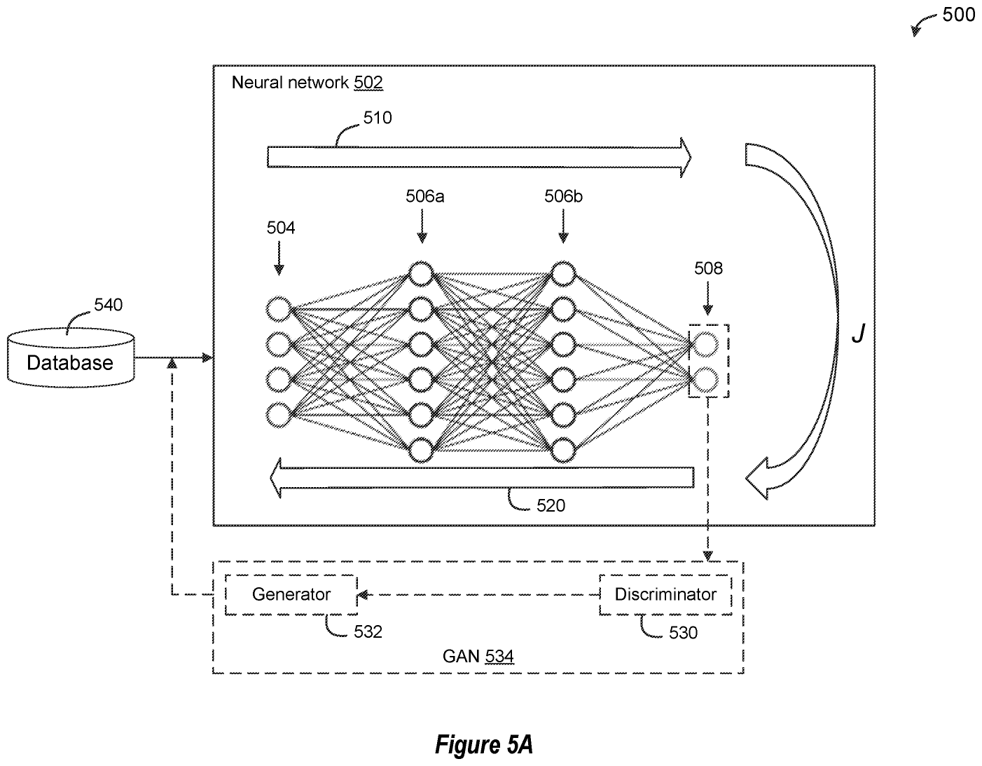

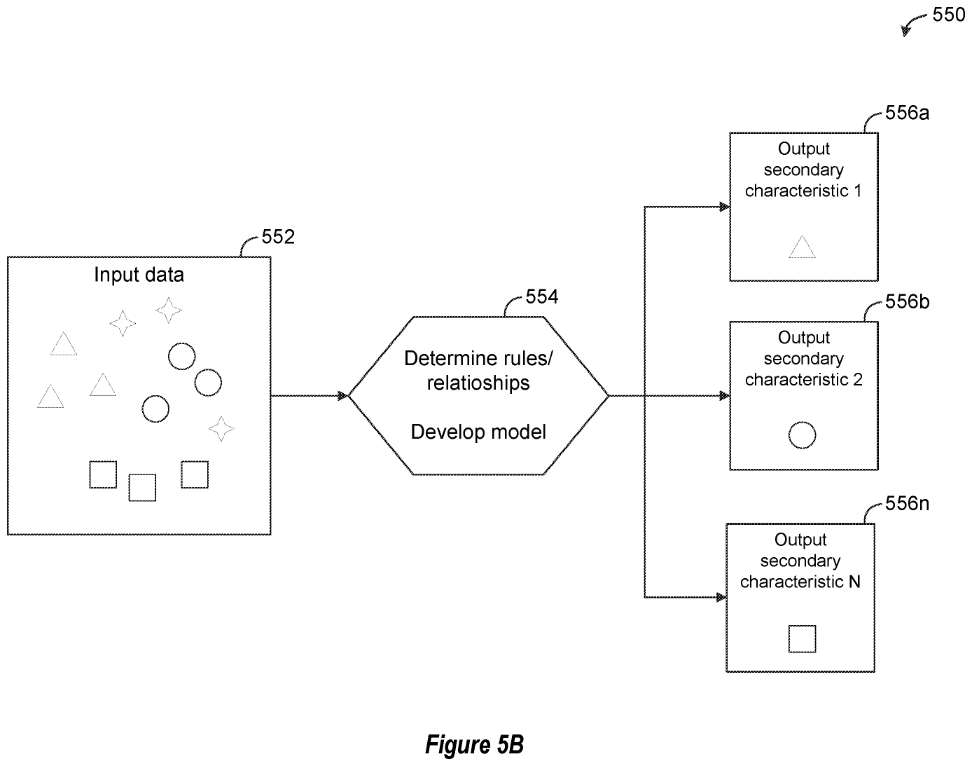

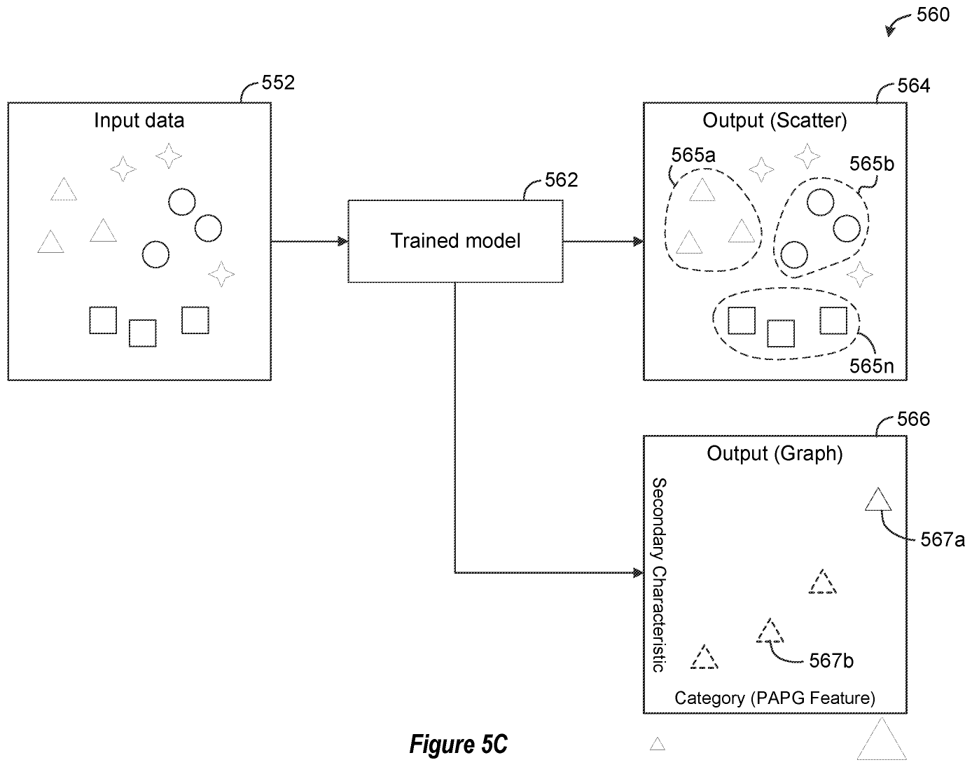

FIG. 5A is a block diagram illustrating a mechanism for training a machine learning model, according to some embodiments. In some embodiments, the training of the machine learning model may be performed by a training module 500. The training module 500 may be embodied in hardware and/or software (e.g., set of instructions and/or code stored on a non-transitory computer-readable storage medium) and implemented by a computerized apparatus or system, which may be implemented separately (e.g., on a workstation) or as part of sensor apparatus 200. In some implementations, the computerized apparatus or system may be part of a cloud computing system (e.g., remote server configured to be communicative with the workstation or sensor apparatus 200), edge computing system, or a hybrid. The training module 500 may include a neural network 502. According to some implementations, neural network 502 may include an input layer 504, an output layer 508, and one or more intermediate “hidden” layers 506a, 506b between the input and output layers. In some implementations, hidden layers may not be present between the input and output layers.

The neural network 502 may represent an algorithm, represented by the layers. Each layer may include one or more nodes, each of which may contain a value or represent a computational function that has one or more weighted input connections, a transformation function that combines the inputs in some way, and/or one or more output connections (which may in turn be input connections to other nodes). The input layer 504 may be configured to receive external data. The external data may be training data from a database 540. In some implementations, a portion (e.g., 20%) of the training data may be randomly selected to be used as part of a validation set for the machine learning model. Each of the hidden layers 506a, 506b may be configured to perform at least a transformation on the inputs. The output layer 508 may be configured to produce a result of the transformations. In some implementations, the result may include predicted data, e.g., a secondary characteristic of a user. The process of producing an output from the input may be referred to as forward propagation 510.

As a training example, one or more nodes of the input layer 504 may receive photoacoustic data that has been categorized by feature, e.g., physiological characteristic of a blood vessel. For instance, an input node 504 may receive and/or store a stiffness, strain, or stress of the blood vessel. One or more hidden layers 506 may receive portions of the stiffness, strain, or stress data, apply one or more weights associated with a given connection, and produce a training output that contains a predicted secondary characteristic. A correlation may exist between the stiffness, strain, or stress data and the predicted secondary characteristic. Such a correlation may be previously unknown. Unsupervised learning may be implemented with the training module 500 to identify the correlation based on input data (as opposed to supervised learning in which the relationship between input and output is already identified and “labeled” for training).

In some embodiments, a modeling process may be performed, e.g., a linear regression, to improve the predictions by the machine learning model. In some embodiments, the modeling process may be logistic regression, which may determine a probability of an outcome given an input, useful for classifying an output (e.g., yes or no, 1 or 0). In linear or logistic regression, an error (J) of the output data (e.g., predicted secondary characteristic) may be determined, and minimized using an optimization technique such as gradient descent via a loss function. In gradient descent, the error is sought to be lowered at each iterative step until a minimum error is reached. In some implementations, linearization may be performed to reduce dimensions and/or learning rate may be set and/or adjusted. In some cases, a learning rate schedule may be set to vary the learning rate to reach the global minimum error without running into nonconvergence from an overly large learning rate or being stuck in local minimum from an overly small learning rate. The process of updating the weights of the connections in the neural network 502 based on the optimization process may be referred to as backpropagation 520.

Forward propagation 510 may then be performed again with the updated connection weights, with another backpropagation 520 based thereon. This cycle may be performed one or more times by the training module 500, e.g., until error is minimized. This training process may thereby result in generation of an optimized machine learning model. In some implementations, the output or the training output may include a prediction with minimized error and high prediction accuracy of, e.g., a secondary characteristic. In some implementations, the output or the training output may contain other data relating to the input, e.g., predicted stiffness, strain, or stress at a future time horizon.

Referring briefly to FIG. 5B, a block diagram representing an example unsupervised learning process 550 is illustrated. Input data 552 may be provided with different types or categories of data represented by triangles, circles, squares, and stars. In some cases, more categories may be present, but for simplicity, only the foregoing are shown. In some cases, these different categories may be known (and also provided as input data 552). In some cases, the categories may not be known. In the illustrated example unsupervised learning process 550, correlation between these data categories and secondary characteristics may not be known. However, through a training process 554, rules (relationships between the categories and secondary characteristics), if any, such as 556a, 556b, 556n, may be identified. The aforementioned training process using forward propagation 510 and backpropagation 520 may be utilized in some approaches of the training process 554 to find relationships within the data and group data points based on the input data alone. A model may thereby be developed through the training process. In some situations, the input data 552 may contain data that do not have correlation with any other type of metric or characteristic, such as the stars. In some cases, none of the categories (including the triangles, circles, and squares) may have a correlation.

FIG. 5C is a block diagram showing a trained model 562, which may be an example of the model developed via the example unsupervised learning process 550 of FIG. 5B. The trained model 562 may be configured to receive input data 552 and output one or more predictions relating to secondary characteristics. In some implementations, a scatter plot 564 may be output, in which categories are clustered into similar groups 565a, 565b, 565n. In some implementations, a graph 566 may be output, in which a category may be plotted against a secondary characteristic. For instance, with a high strain detected based on PAPG signal input data, it may be determined that there is a correlation to a location, or close distance or high proximity to a high-pollution (e.g., high-AQI) area, indicated by a solid triangle 567a. With lower strain, there may be a correlation to a lower proximity to the high-pollution area (indicated by a dashed triangle 567b). Hence, unsupervised learning can cluster data points without being provided the relationship.

Referring back to FIG. 5A, in some embodiments, additional input data may be utilized with the neural network 502. More specifically, a discriminator 530 and a generator 532 may optionally be implemented with the neural network 502. A discriminator is a type of neural network configured to learn to distinguish fake data from realistic fake data that may have the same characteristics as the training data and generated by the generator 532. The discriminator 530 and the generator 532 may compete with each other, and the discriminator 530 may penalize the generator 532 for generating data that is easily recognized as implausible. By using the discriminator 530 and the generator 532 together in such a way in a generative adversarial network (GAN) 534, more realistic and plausible examples may be generated by the generator 532 over time. In this way, in some embodiments, data in addition to those collected may be used for training.

In some embodiments, the resulting output may include values included in a predefined format such as those noted elsewhere above. The resulting output may be used in various ways. For instance, in some cases, the output may be stored in a storage device. In some cases, the output may be displayed. In some cases, the output may be used as input for another training process. In some embodiments, the resulting output may include cluster data that indicates a grouping of secondary characteristics, e.g., on a scatter plot of a secondary characteristic (e.g., user location or distance from a reference location) with respect to PAPG features (e.g., stiffness, strain, or stress). Such cluster data may represent a collection of predictions.

In some embodiments, the training of the machine learning model may be performed at a computerized system and applied at a sensor apparatus 200. In some embodiments, the training may be performed and applied at the sensor apparatus 200.

That is, inference (e.g., output of prediction based on input data) based on a trained machine learning model may be performed at sensor apparatus 200. In some implementations, the resulting output generated by the sensor apparatus 200 may be sent to another device, e.g., another sensor apparatus, or a local or remote computerized device or system (e.g., workstation, server, storage device, display device). In some embodiments, inference may be performed at the sensor apparatus 200 based on a trained model received from a computerized system. The resulting output may be further evaluated, or otherwise stored or displayed.

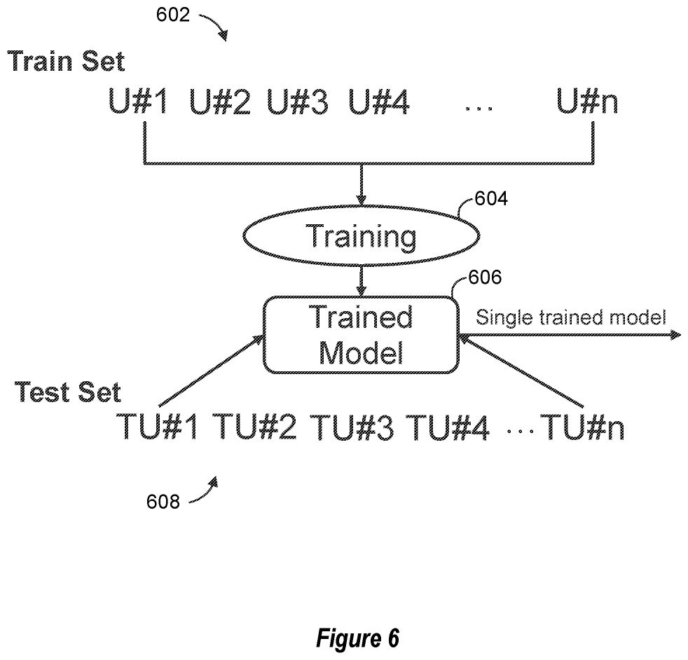

FIG. 6 shows an example generation and use of a single trained model. In training and developing a machine learning (ML) model, a training set 602 of user data (e.g., of User 1 through User N) or portion(s) thereof may be input to a training process 604. The training process 604 may be an example of the training process illustrated and discussed with respect to FIG. 5A. In some examples, the training process 604 may be performed by training module 500. As a result of the training process 604, a trained machine learning model 606 may be generated. Such a trained machine learning model 606 may be configured to output a prediction according to the training process 604 (e.g., weights adjusted during the training process 604).

During inference of the trained machine learning model 606, a test set 608 of user data (e.g., of Test User 1 through Test User N) or portion(s) thereof may be input to the trained machine learning model 606. Based on the input test set 608, the trained machine learning model 606 may produce a prediction. In this example, a single trained model is being used.

However, a single ML model may be limited to one (or at most few) types of input data and output data. Consider photoacoustic (PAPG) signals, e.g., obtained from sensor apparatus 200, which have a large amount of information that can be leveraged, as multiple types of parameters, metrics, and characteristics can be derived from the signal data. Moreover, despite the great number of possibilities of insights that can be determined from physiological data from a single target object (e.g., blood vessel), biometrics can also be influenced by other parts of a user's body (e.g., muscles, tendons) and behaviors of a user (e.g., mechanical movement of limbs detectable by an inertial sensor such as gyroscope or accelerometer, movement through an area, physical activity detected by an activity tracker). Thus, some level of categorization and identification of multiple categories used in conjunction with multiple corresponding ML models can greatly enhance the performance of a given model or models collectively (e.g., if implemented in concert as an ensemble model).

Given the wide range of data types and categories that can be extracted from certain types of signals such as PAPG signals or other biometric signals, specialized, personalized, or tailored ML models based on groups of users can be built to advantageously obtain an equally wide range of insights and even yet-unknown correlations and insights, which can be used in various applications, including health and wellness monitoring and supporting population studies.

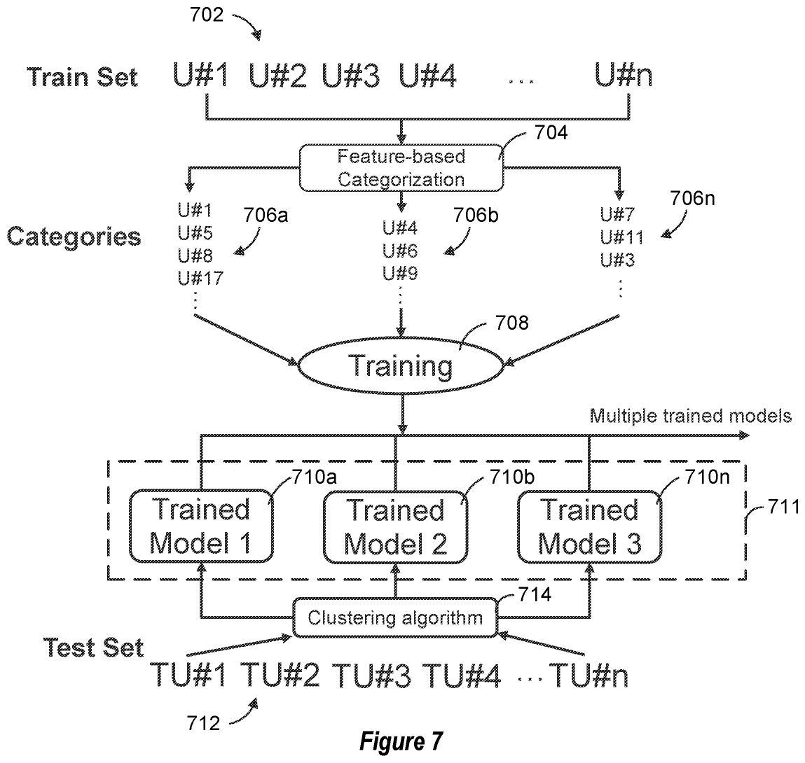

To the above ends, FIG. 7 shows an example generation and use of multiple trained models, according to some embodiments. In some cases, the multiple trained models may include category-based machine learning (ML) models. That is, according to some examples, a training set 702 of user data (e.g., of User 1 through User N) or portion(s) thereof may be categorized based on feature (e.g., at block 704).

Features may include physiological characteristics of a target object (e.g., blood vessel) of a user and/or physiological parameters of the user. Some examples of physiological characteristics may include (but are not limited to) stiffness, strain, stress, distension, compliance, dimension (e.g., diameter, cross-sectional area), PWV, and other indication of deformation or force applied to a blood vessel of a user. Some examples of physiological parameters of the user may include (but are not limited to) a blood pressure, a blood glucose level, a blood oxygen level, an obesity level (e.g., body fat) of the user. Features indicative of healthiness of a user can also be identified, e.g., non-stiff arteries. Physiological characteristics may be determined from biometric (e.g., photoacoustic) signals detected, e.g., by sensor apparatus 200. Physiological parameters may be derived from the physiological characteristics, e.g., based on known relationships between the characteristics and the parameters. As an example, blood pressure may be estimated based on PWV.

However, using the approaches described herein, biometric (e.g., photoacoustic) signals need not be limited to determination of PWV or blood pressure. Arterial characteristics need not be limited to derivation of physiological parameters. Secondary characteristics such as user location, habits (e.g., smoking), activities, etc. may be determined.

In some embodiments, the training set 702 may be grouped into multiple categories (e.g., 706a, 706b, 706n) of user data based on multiple features. A first feature may be arterial stiffness. A second feature may be arterial strain. A third feature may be arterial stress. These features may be determined based on photoacoustic signals included with the training set 702 of user data. For instance, in some approaches, fluid mechanics concepts can be leveraged to calculate stiffness and strain once the diameter and distension of an artery are extracted from raw photoacoustic signals (e.g., waveforms) or photoacoustic images. Strain may be defined by Δr/r, where r is the radius of the artery. Arterial stiffness may be defined by (ΔP·r²)/Δr, where ΔP (change in pressure) is based on a training ground truth pressure measurement. Any changes in arterial stiffness can also reflected in a PWV measurement that can be made. Arterial shear stress may be defined by μ·Δu/Δy, where μ is the fluid viscosity of blood, u is the fluid velocity of the blood, and y is the distance from the surface (which can be r in the center of the artery).

In some embodiments, the multiple categories 706a, 706b, 706n of user data, which may be categorized based on respective features as mentioned above, may be inputted into a training process 708. The training process 708 may be an example of the training process illustrated and discussed with respect to FIG. 5A. In some examples, the training process 708 may be performed by training module 500. In some implementations, the training process 708 may be applied to each category 706a, 706b, 706n of user data separately. That is, the training process 708 may be applied three times, which may done concurrently, in parallel, or sequentially. As a result of the training process 708 (or multiple processes), multiple trained machine learning models 710a, 710b, 710n corresponding to respective categories 706a, 706b, 706n of user data may be generated. In some cases, such trained machine learning models 710a, 710b, 710n may each be configured to output a different prediction according to the training process 708 (e.g., weights adjusted during the training process 708). It will be appreciated that more or fewer categories than the three shown may be determined and sent to the training process 708, and more or fewer trained machine learning models 710a, 710b, 710n may be generated.

In some cases, an ensemble model 711 may be created with all or multiple ones of the trained machine learning models 710a, 710b, 710n. An ensemble model may refer to multiple models collectively used to predict an outcome. The multiple models may be generated by using, for example, different categories, different training data sets, or different modeling algorithms (e.g., different learning rates, weights, stopping points).

In some configurations, within an ensemble model of two (or more) trained ML models, a first trained ML model may take, as input, an output of a second trained ML model. In this way, multiple categories or feature types may be considered. For instance, it may be found that a user or group of users having both high arterial stress and high arterial strain is predicted with accuracy to live in a high-pollution area or have certain smoking habits, while an individual ML model trained to receive data indicative of either high arterial stress or high arterial strain alone may not output such a prediction or produce a prediction that has lower accuracy.

In some configurations, an ensemble model may aggregate (e.g., use an average, a weighted combination, or other ways) the prediction of each constituent model and produce one prediction. In these configurations, two or more of the trained machine learning models 710a, 710b, 710n may be configured to predict the same secondary characteristic (e.g., strain). An ensemble model 711 with those two or more trained ML models may generate a prediction of that same secondary characteristic (e.g., strain) which is more accurate than a prediction from one of the trained ML models.

During inference, once the trained machine learning models 710a, 710b, 710n are generated, a test set 712 of user data (e.g., of Test User 1 through Test User N) or portion(s) thereof may be input to the trained models. The test set 712 may include, for example, physiological (e.g., photoacoustic) signals associated with various users. In some implementations, a clustering algorithm 714 may identify groups of user categories or physiological features within the signals provided the test set 712. In some cases, a non-automatic approach may be taken in which groups of user categories are identified or labeled manually.

In some cases, the clustering algorithm 714 may be part of one or more of the trained machine learning models 710a, 710b, 710n or the ensemble model 711. In some cases, the clustering algorithm 714 may be a trained ML model configured to organize the test set 712 into clusters. Examples of clustering algorithms may include centroid-based (e.g., K-means) clustering, density-based clustering, distribution-based (e.g., Gaussian mixture model (GMM)) clustering, and others known to those with ordinary skill in the relevant arts.

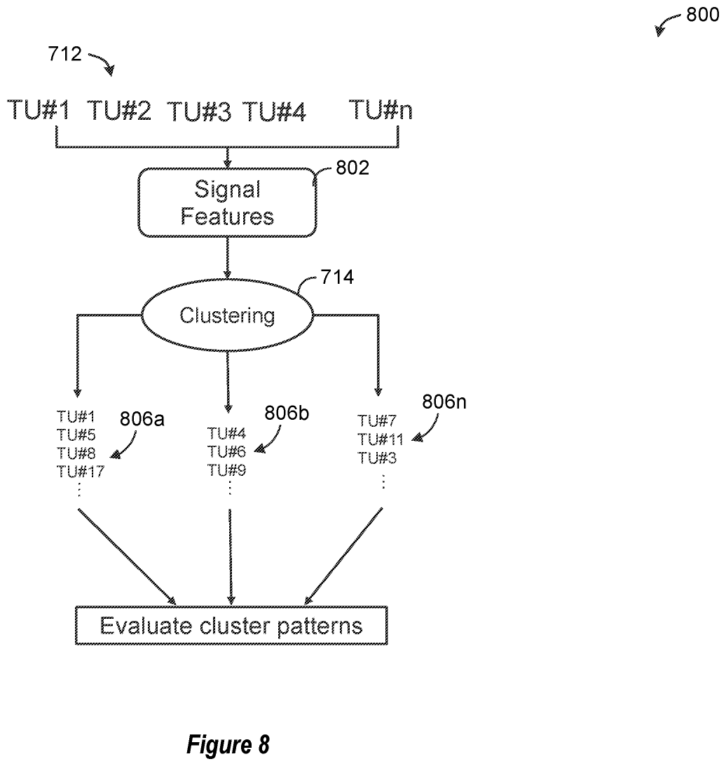

Referring briefly to FIG. 8, a more specific example clustering process 800 for inference with multiple trained models is shown, according to some embodiments. In some examples, features 802 may be extracted from physiological (e.g., photoacoustic) signals associated with various users who are part of the test set 712 of user data. In some implementations, a clustering algorithm 714, which may be at least one of the above examples, may be used to organize the test set 712 of user data into categories 806a, 806b, 806n. In some approaches, the categories 806a, 806b, 806n of user data may then be fed into (or where the clustering algorithm 714 is part of a ML model, used by) one or more of the trained machine learning models 710a, 710b, 710n or the ensemble model 711 to generate a prediction, e.g., of one or more secondary characteristics associated with the users.

Returning back to FIG. 7, identified groups of the test set 712 may be input to respective one(s) of the trained machine learning models 710a, 710b, 710n, or to the ensemble model 711. Based on the input, the trained machine learning models 710a, 710b, 710n or the ensemble model 711 may produce one or more predictions, e.g., of one or more secondary characteristics associated with the users.

### Example Methods

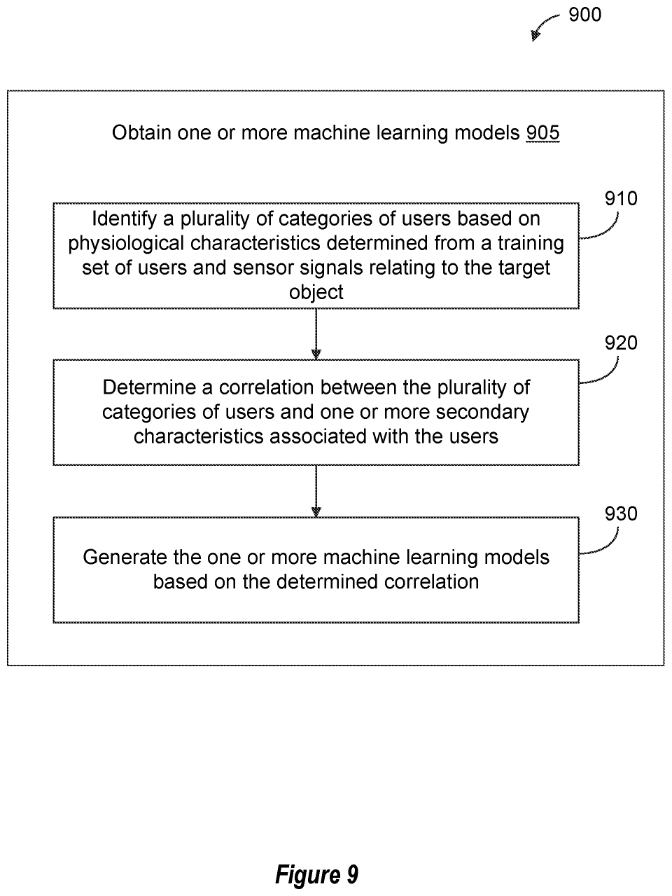

FIG. 9 is a flow diagram of a method 900 of obtaining one or more machine learning models configured to predict a user characteristic, according to some embodiments. Structure for performing the functionality illustrated in one or more of the blocks shown in FIG. 9 may include hardware and/or software components of a computerized apparatus or system. Components of such apparatus or system may include, for example, a control system (including one or more processors), a memory, and/or a computer-readable apparatus including a storage medium storing computer-readable and/or computer-executable instructions that are configured to, when executed by the control system, cause the control system, the one or more processors, or the apparatus to perform operations represented by blocks below. In some cases, the blocks of FIG. 9 may be performed by, for example, the sensor apparatus 200 or a similar apparatus, or a component thereof (e.g., a control system).

The method outlined in FIG. 9 may include more or fewer blocks than indicated. Moreover, the blocks of methods disclosed herein are not necessarily performed in the order indicated. In some instances, one or more of the blocks shown in FIG. 9 may be performed concurrently.

At block 905, the method 900 may include obtaining one or more machine learning models according to blocks 910-930 described below.

At block 910, the method 900 may include identifying a plurality of categories of users based on physiological characteristics determined from a training set of users and sensor signals relating to a target object of the users. In some embodiments, the sensor signals of the training set may include a training set of photoacoustic signals; and the determining of the correlation may include determining a correlation between the plurality of categories of users and an unknown secondary characteristic associated with the users.

In some embodiments, the target object of the users may include a blood vessel. Examples of the physiological characteristics may include arterial stiffness, strain, stress, distension, compliance, dimension(s), pulse wave velocity (PWV), and/or other indication of deformation or force applied to a blood vessel of a user. Such physiological characteristics may be determined based on, for example, analyses of HRW features and/or images obtained from the photoacoustic signals. A diameter of a blood vessel and/or an arterial cross-sectional area (which may be examples of dimensions) may be determined from the analysis of a photoacoustic image, for instance, and other characteristics may be estimated based thereon. Various physiological characteristics may be derived from this information as mentioned above; see, e.g., formulas for arterial stiffness, strain, and stress.

At block 920, the method 900 may include determining a correlation between the plurality of categories of users and one or more secondary characteristics associated with the users. In some embodiments, the determining of the correlation between the plurality of categories of users and the one or more secondary characteristics associated with the users may include an unsupervised learning process. This unsupervised learning process may be an example of that discussed with respect to FIGS. 5-5B.

At block 930, the method 900 may include generating the one or more machine learning models based on the determined correlation. In some embodiments, the one or more machine learning models may include at least a first model and a second model that correlate to respective ones of the plurality of categories of users; the first model may be configured to predict a first secondary characteristic associated with the user based on the at least one physiological characteristic associated with the user; and the second model may be configured to predict a second secondary characteristic associated with the user based on the at least one physiological characteristic associated with the user. In some variations, the one or more machine learning models may include an ensemble model configured to predict the at least one secondary characteristic associated with the user using both the first model and the second model.

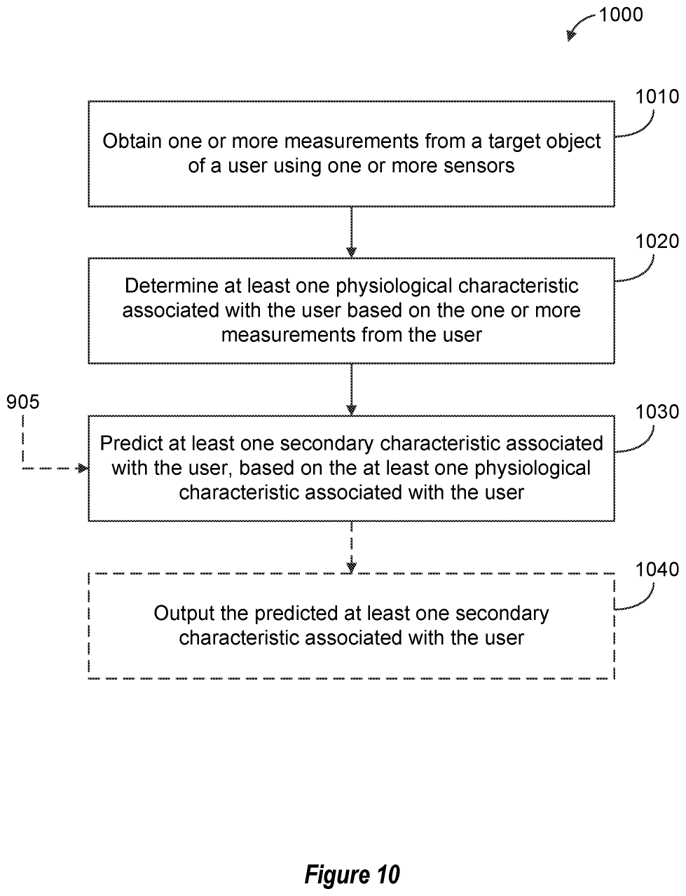

FIG. 10 is a flow diagram of a method 1000 of predicting a user characteristic using a category-based model, according to some embodiments. Structure for performing the functionality illustrated in one or more of the blocks shown in FIG. 10 may include hardware and/or software components of a computerized apparatus or system (which may be implemented as a wearable device in some embodiments). Components of such apparatus or system may include, for example, a light source system, a receiver system, a control system (including one or more processors), a memory, and/or a computer-readable apparatus including a storage medium storing computer-readable and/or computer-executable instructions that are configured to, when executed by the control system, cause the control system, the one or more processors, or the apparatus to perform operations represented by blocks below. Example components of the apparatus are illustrated in, e.g., FIG. 2, which are described in more detail above. In some embodiments, the blocks of FIG. 10 may be performed by, for example, the sensor apparatus 200 or a similar apparatus, or a component thereof (e.g., a control system).

The method outlined in FIG. 10 may include more or fewer blocks than indicated. Moreover, the blocks of methods disclosed herein are not necessarily performed in the order indicated. In some instances, one or more of the blocks shown in FIG. 9 may be performed concurrently.

At block 1010, the method 1000 may include obtaining, by a control system, one or more measurements from a target object of a user using one or more sensors. In some embodiments, the one or more sensors may be configured to receive photoacoustic signals from the target object of the user.

At block 1020, the method 1000 may include determining, by the control system, at least one physiological characteristic associated with the user based on the one or more measurements from the user. In some embodiments, the target object of the user may include a blood vessel of the user; and the at least one physiological characteristic associated with the user may include a strain of the blood vessel, a stress of the blood vessel, a distension of the blood vessel, a stiffness of the blood vessel, a compliance of the blood vessel, a dimension of the blood vessel, or a combination thereof. In some embodiments, the at least one physiological characteristic associated with the user may include a blood pressure of the user.

At block 1030, the method 1000 may include predicting, by the control system, at least one secondary characteristic associated with the user, based on the at least one physiological characteristic associated with the user. In some embodiments, the predicted at least one second characteristic associated with the user may relate to a location of the user, a metric associated with the location of the user, a behavioral pattern of the user, or a combination thereof. In some embodiments, the predicting of the at least one secondary characteristic associated with the user may include using one or more machine learning models implemented by the control system. In some implementations, the one or more machine learning models may be obtained according to block 905 of FIG. 9.

In some embodiments, at block 1040, the method 1000 may optionally include outputting, by the control system, the predicted at least one secondary characteristic associated with the user. In further embodiments, the outputted prediction may be stored, displayed, sent to another computerized device, or otherwise used in a downstream application.

As used herein, a phrase referring to “at least one of” a list of items refers to any combination of those items, including single members. As an example, “at least one of: a, b, or c” is intended to cover: a, b, c, a-b, a-c, b-c, and a-b-c.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described above. Whether such functionality is implemented in hardware or software depends upon the particular application and design constraints imposed on the overall system.

The hardware and data processing apparatus used to implement the various illustrative logics, logical blocks, modules and circuits described in connection with the aspects disclosed herein may be implemented or performed with a general purpose single- or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, such as a combination of a DSP and a microprocessor, multiple microprocessors, one or more microprocessors in conjunction with a DSP core, or any other such configuration. In some implementations, particular processes and methods may be performed by circuitry that is specific to a given function.

In one or more aspects, the functions described may be implemented in hardware, digital electronic circuitry, computer software, firmware, including the structures disclosed in this specification and their structural equivalents thereof, or in any combination thereof. Implementations of the subject matter described in this specification also may be implemented as one or more computer programs, i.e., one or more modules of computer program instructions, encoded on a computer storage media for execution by, or to control the operation of, data processing apparatus.

If implemented in software, the functions may be stored on or transmitted over as one or more instructions or code on a computer-readable medium, such as a non-transitory medium. The processes of a method or algorithm disclosed herein may be implemented in a processor-executable software module which may reside on a computer-readable medium. Computer-readable media include both computer storage media and communication media including any medium that may be enabled to transfer a computer program from one place to another. Storage media may be any available media that may be accessed by a computer. By way of example, and not limitation, non-transitory media may include RAM, ROM, EEPROM, CD-ROM or other optical disk storage, magnetic disk storage or other magnetic storage devices, or any other medium that may be used to store desired program code in the form of instructions or data structures and that may be accessed by a computer. Also, any connection may be properly termed a computer-readable medium. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and instructions on a machine readable medium and computer-readable medium, which may be incorporated into a computer program product.

Various modifications to the implementations described in this disclosure may be readily apparent to a person having ordinary skill in the art, and the generic principles defined herein may be applied to other implementations without departing from the spirit or scope of this disclosure. Thus, the disclosure is not intended to be limited to the implementations shown herein, but is to be accorded the widest scope consistent with the claims, the principles and the novel features disclosed herein. The word “exemplary” is used exclusively herein, if at all, to mean “serving as an example, instance, or illustration.” Any implementation described herein as “exemplary” is not necessarily to be construed as preferred or advantageous over other implementations.

Certain features that are described in this specification in the context of separate implementations also may be implemented in combination in a single implementation. Conversely, various features that are described in the context of a single implementation also may be implemented in multiple implementations separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products. Additionally, other implementations are within the scope of the following claims. In some cases, the actions recited in the claims may be performed in a different order and still achieve desirable results.

It will be understood that unless features in any of the particular described implementations are expressly identified as incompatible with one another or the surrounding context implies that they are mutually exclusive and not readily combinable in a complementary and/or supportive sense, the totality of this disclosure contemplates and envisions that specific features of those complementary implementations may be selectively combined to provide one or more comprehensive, but slightly different, technical solutions. It will therefore be further appreciated that the above description has been given by way of example only and that modifications in detail may be made within the scope of this disclosure.

Various modifications to the implementations described in this disclosure may be readily apparent to a person having ordinary skill in the art, and the generic principles defined herein may be applied to other implementations without departing from the spirit or scope of this disclosure. Thus, the following claims are not intended to be limited to the implementations shown herein, but are to be accorded the widest scope consistent with this disclosure, the principles and the novel features disclosed herein.

Additionally, certain features that are described in this specification in the context of separate implementations also can be implemented in combination in a single implementation. Conversely, various features that are described in the context of a single implementation also can be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one more example processes in the form of a flow diagram. However, other operations that are not depicted can be incorporated in the example processes that are schematically illustrated. For example, one or more additional operations can be performed before, after, simultaneously, or between any of the illustrated operations. Moreover, various ones of the described and illustrated operations can itself include and collectively refer to a number of sub-operations. For example, each of the operations described above can itself involve the execution of a process or algorithm. Furthermore, various ones of the described and illustrated operations can be combined or performed in parallel in some implementations. Similarly, the separation of various system components in the implementations described above should not be understood as requiring such separation in all implementations. As such, other implementations are within the scope of the following claims. In some cases, the actions recited in the claims can be performed in a different order and still achieve desirable results.

Implementation examples are described in the following numbered clauses:

Clause 1. A method of predicting a user characteristic using a category-based model, the method comprising: obtaining, by a control system, one or more measurements from a target object of a user using one or more sensors; determining, by the control system, at least one physiological characteristic associated with the user based on the one or more measurements from the user; predicting, by the control system, at least one secondary characteristic associated with the user, based on the at least one physiological characteristic associated with the user; and outputting, by the control system, the predicted at least one secondary characteristic associated with the user.

Clause 2. The method of clause 1, wherein the predicted at least one second characteristic associated with the user relates to a location of the user, a metric associated with the location of the user, a behavioral pattern of the user, or a combination thereof.

Clause 3. The method of clause 1, wherein the one or more sensors are configured to receive photoacoustic signals from the target object of the user.

Clause 4. The method of clause 1, wherein: the target object of the user comprises a blood vessel of the user; and the at least one physiological characteristic associated with the user comprises a strain of the blood vessel, a stress of the blood vessel, a distension of the blood vessel, a stiffness of the blood vessel, a compliance of the blood vessel, a dimension of the blood vessel, or a combination thereof.

Clause 5. The method of clause 1, wherein the at least one physiological characteristic associated with the user comprises a blood pressure of the user.

Clause 6. The method of clause 1, wherein the predicting of the at least one secondary characteristic associated with the user comprises using one or more machine learning models implemented by the control system, the one or more machine learning models obtained by: identifying a plurality of categories of users based on physiological characteristics determined from a training set of users and sensor signals relating to a target object of the users; determining a correlation between the plurality of categories of users and one or more secondary characteristics associated with the users; and generating the one or more machine learning models based on the determined correlation.

Clause 7. The method of clause 6, wherein: the sensor signals of the training set comprise a training set of photoacoustic signals; and the determining of the correlation comprises determining a correlation between the plurality of categories of users and an unknown secondary characteristic associated with the users.

Clause 8. The method of clause 6, wherein the determining of the correlation between the plurality of categories of users and the one or more secondary characteristics associated with the users comprises an unsupervised learning process.

Clause 9. The method of clause 6, wherein: the one or more machine learning models comprise at least a first model and a second model that correlate to respective ones of the plurality of categories of users; the first model is configured to predict a first secondary characteristic associated with the user based on the at least one physiological characteristic associated with the user; and the second model is configured to predict a second secondary characteristic associated with the user based on the at least one physiological characteristic associated with the user.

Clause 10. The method of clause 9, wherein the one or more machine learning models comprise an ensemble model configured to predict the at least one secondary characteristic associated with the user using both the first model and the second model.

Clause 11. An apparatus comprising: one or more sensors; and a control system comprising one or more processors configured to: obtain one or more measurements from a target object of a user using the one or more sensors; determine at least one physiological characteristic associated with the user based on the one or more measurements from the user; predict at least one secondary characteristic associated with the user, based on the at least one physiological characteristic associated with the user; and output the predicted at least one secondary characteristic associated with the user.

Clause 12. The apparatus of clause 11, wherein the predicted at least one second characteristic associated with the user relates to a location of the user, a metric associated with the location of the user, a behavioral pattern of the user, or a combination thereof.

Clause 13. The apparatus of clause 11, wherein the one or more sensors are configured to receive photoacoustic signals from the target object of the user.

Clause 14. The apparatus of clause 11, wherein: the target object of the user comprises a blood vessel of the user; and the at least one physiological characteristic associated with the user comprises a strain of the blood vessel, a stress of the blood vessel, a distension of the blood vessel, a stiffness of the blood vessel, a compliance of the blood vessel, a dimension of the blood vessel, or a combination thereof.

Clause 15. The apparatus of clause 11, wherein the at least one physiological characteristic associated with the user comprises a blood pressure of the user.

Clause 16. The apparatus of clause 11, wherein the predicting of the at least one secondary characteristic associated with the user comprises using one or more machine learning models implemented by the control system, the one or more machine learning models obtained by: identifying a plurality of categories of users based on physiological characteristics determined from a training set of users and sensor signals relating to a target object of the users; determining a correlation between the plurality of categories of users and one or more secondary characteristics associated with the users; and generating the one or more machine learning models based on the determined correlation.

Clause 17. The apparatus of clause 16, wherein: the sensor signals of the training set comprise a training set of photoacoustic signals; and the determining of the correlation comprises determining a correlation between the plurality of categories of users and an unknown secondary characteristic associated with the users.

Clause 18. The apparatus of clause 16, wherein the determining of the correlation between the plurality of categories of users and the one or more secondary characteristics associated with the users comprises an unsupervised learning process.

Clause 19. An apparatus comprising: means for obtaining one or more measurements from a target object of a user using one or more sensors; means for determining at least one physiological characteristic associated with the user based on the one or more measurements from the user; means for predicting at least one secondary characteristic associated with the user, based on the at least one physiological characteristic associated with the user; and means for outputting the predicted at least one secondary characteristic associated with the user.

Clause 20. The apparatus of clause 19, wherein the predicted at least one second characteristic associated with the user relates to a location of the user, a metric associated with the location of the user, a behavioral pattern of the user, or a combination thereof.

Clause 21. The apparatus of clause 19, wherein the one or more sensors are configured to receive photoacoustic signals from the target object of the user.

Clause 22. The apparatus of clause 19, wherein: the target object of the user comprises a blood vessel of the user; and the at least one physiological characteristic associated with the user comprises a strain of the blood vessel, a stress of the blood vessel, a distension of the blood vessel, a stiffness of the blood vessel, a compliance of the blood vessel, a dimension of the blood vessel, or a combination thereof.

Clause 23. The apparatus of clause 19, wherein the at least one physiological characteristic associated with the user comprises a blood pressure of the user.

Clause 24. The apparatus of clause 19, wherein the predicting of the at least one secondary characteristic associated with the user comprises using one or more machine learning models implemented by a control system, the one or more machine learning models obtained using: means for identifying a plurality of categories of users based on physiological characteristics determined from a training set of users and sensor signals relating to a target object of the users; means for determining a correlation between the plurality of categories of users and one or more secondary characteristics associated with the users; and means for generating the one or more machine learning models based on the determined correlation.

Clause 25. A non-transitory computer-readable apparatus comprising a storage medium, the storage medium comprising a plurality of instructions configured to, when executed by one or more processors of a control system, cause an apparatus to: obtain, by the control system, one or more measurements from a target object of a user using one or more sensors; determine, by the control system, at least one physiological characteristic associated with the user based on the one or more measurements from the user; predict, by the control system, at least one secondary characteristic associated with the user, based on the at least one physiological characteristic associated with the user; and output, by the control system, the predicted at least one secondary characteristic associated with the user.

Clause 26. The non-transitory computer-readable apparatus of clause 25, wherein the predicted at least one second characteristic associated with the user relates to a location of the user, a metric associated with the location of the user, a behavioral pattern of the user, or a combination thereof.

Clause 27. The non-transitory computer-readable apparatus of clause 25, wherein the one or more sensors are configured to receive photoacoustic signals from the target object of the user.

Clause 28. The non-transitory computer-readable apparatus of clause 25, wherein: the target object of the user comprises a blood vessel of the user; and the at least one physiological characteristic associated with the user comprises a strain of the blood vessel, a stress of the blood vessel, a distension of the blood vessel, a stiffness of the blood vessel, a compliance of the blood vessel, a dimension of the blood vessel, or a combination thereof.

Clause 29. The non-transitory computer-readable apparatus of clause 25, wherein the at least one physiological characteristic associated with the user comprises a blood pressure of the user.

Clause 30. The non-transitory computer-readable apparatus of clause 25, wherein the predicting of the at least one secondary characteristic associated with the user comprises using one or more machine learning models implemented by the control system, the one or more machine learning models obtained by: identifying a plurality of categories of users based on physiological characteristics determined from a training set of users and sensor signals relating to a target object of the users; determining a correlation between the plurality of categories of users and one or more secondary characteristics associated with the users; and generating the one or more machine learning models based on the determined correlation.

What is claimed:

1. A method of predicting a user characteristic using a category-based model, the method comprising:
obtaining, by a control system, one or more measurements from a target object of a user using one or more sensors;
determining, by the control system, at least one physiological characteristic associated with the user based on the one or more measurements from the user;
predicting, by the control system, at least one secondary characteristic associated with the user, based on the at least one physiological characteristic associated with the user; and
outputting, by the control system, the predicted at least one secondary characteristic associated with the user.

2. The method of claim 1, wherein the predicted at least one second characteristic associated with the user relates to a location of the user, a metric associated with the location of the user, a behavioral pattern of the user, or a combination thereof.

3. The method of claim 1, wherein the one or more sensors are configured to receive photoacoustic signals from the target object of the user.

4. The method of claim 1, wherein:
the target object of the user comprises a blood vessel of the user; and
the at least one physiological characteristic associated with the user comprises a strain of the blood vessel, a stress of the blood vessel, a distension of the blood vessel, a stiffness of the blood vessel, a compliance of the blood vessel, a dimension of the blood vessel, or a combination thereof.

5. The method of claim 1, wherein the at least one physiological characteristic associated with the user comprises a blood pressure of the user.

6. The method of claim 1, wherein the predicting of the at least one secondary characteristic associated with the user comprises using one or more machine learning models implemented by the control system, the one or more machine learning models obtained by:
identifying a plurality of categories of users based on physiological characteristics determined from a training set of users and sensor signals relating to a target object of the users;
determining a correlation between the plurality of categories of users and one or more secondary characteristics associated with the users; and
generating the one or more machine learning models based on the determined correlation.

7. The method of claim 6, wherein:
the sensor signals of the training set comprise a training set of photoacoustic signals; and
the determining of the correlation comprises determining a correlation between the plurality of categories of users and an unknown secondary characteristic associated with the users.

8. The method of claim 6, wherein the determining of the correlation between the plurality of categories of users and the one or more secondary characteristics associated with the users comprises an unsupervised learning process.

9. The method of claim 6, wherein:
the one or more machine learning models comprise at least a first model and a second model that correlate to respective ones of the plurality of categories of users;
the first model is configured to predict a first secondary characteristic associated with the user based on the at least one physiological characteristic associated with the user; and
the second model is configured to predict a second secondary characteristic associated with the user based on the at least one physiological characteristic associated with the user.

10. The method of claim 9, wherein the one or more machine learning models comprise an ensemble model configured to predict the at least one secondary characteristic associated with the user using both the first model and the second model.

11. An apparatus comprising:
one or more sensors; and
a control system comprising one or more processors configured to:
obtain one or more measurements from a target object of a user using the one or more sensors;
determine at least one physiological characteristic associated with the user based on the one or more measurements from the user;
predict at least one secondary characteristic associated with the user, based on the at least one physiological characteristic associated with the user; and
output the predicted at least one secondary characteristic associated with the user.

12. The apparatus of claim 11, wherein the predicted at least one second characteristic associated with the user relates to a location of the user, a metric associated with the location of the user, a behavioral pattern of the user, or a combination thereof.

13. The apparatus of claim 11, wherein the one or more sensors are configured to receive photoacoustic signals from the target object of the user.

14. The apparatus of claim 11, wherein:
the target object of the user comprises a blood vessel of the user; and
the at least one physiological characteristic associated with the user comprises a strain of the blood vessel, a stress of the blood vessel, a distension of the blood vessel, a stiffness of the blood vessel, a compliance of the blood vessel, a dimension of the blood vessel, or a combination thereof.

15. The apparatus of claim 11, wherein the at least one physiological characteristic associated with the user comprises a blood pressure of the user.

16. The apparatus of claim 11, wherein the predicting of the at least one secondary characteristic associated with the user comprises using one or more machine learning models implemented by the control system, the one or more machine learning models obtained by:
identifying a plurality of categories of users based on physiological characteristics determined from a training set of users and sensor signals relating to a target object of the users;
determining a correlation between the plurality of categories of users and one or more secondary characteristics associated with the users; and
generating the one or more machine learning models based on the determined correlation.

17. The apparatus of claim 16, wherein:
the sensor signals of the training set comprise a training set of photoacoustic signals; and
the determining of the correlation comprises determining a correlation between the plurality of categories of users and an unknown secondary characteristic associated with the users.

18. The apparatus of claim 16, wherein the determining of the correlation between the plurality of categories of users and the one or more secondary characteristics associated with the users comprises an unsupervised learning process.

19. An apparatus comprising:
means for obtaining one or more measurements from a target object of a user using one or more sensors;
means for determining at least one physiological characteristic associated with the user based on the one or more measurements from the user;
means for predicting at least one secondary characteristic associated with the user, based on the at least one physiological characteristic associated with the user; and
means for outputting the predicted at least one secondary characteristic associated with the user.

20. The apparatus of claim 19, wherein the predicted at least one second characteristic associated with the user relates to a location of the user, a metric associated with the location of the user, a behavioral pattern of the user, or a combination thereof.

21. The apparatus of claim 19, wherein the one or more sensors are configured to receive photoacoustic signals from the target object of the user.

22. The apparatus of claim 19, wherein:
the target object of the user comprises a blood vessel of the user; and
the at least one physiological characteristic associated with the user comprises a strain of the blood vessel, a stress of the blood vessel, a distension of the blood vessel, a stiffness of the blood vessel, a compliance of the blood vessel, a dimension of the blood vessel, or a combination thereof.

23. The apparatus of claim 19, wherein the at least one physiological characteristic associated with the user comprises a blood pressure of the user.

24. The apparatus of claim 19, wherein the predicting of the at least one secondary characteristic associated with the user comprises using one or more machine learning models implemented by a control system, the one or more machine learning models obtained using:
means for identifying a plurality of categories of users based on physiological characteristics determined from a training set of users and sensor signals relating to a target object of the users;
means for determining a correlation between the plurality of categories of users and one or more secondary characteristics associated with the users; and
means for generating the one or more machine learning models based on the determined correlation.

25. A non-transitory computer-readable apparatus comprising a storage medium, the storage medium comprising a plurality of instructions configured to, when executed by one or more processors of a control system, cause an apparatus to:
obtain, by the control system, one or more measurements from a target object of a user using one or more sensors;
determine, by the control system, at least one physiological characteristic associated with the user based on the one or more measurements from the user;
predict, by the control system, at least one secondary characteristic associated with the user, based on the at least one physiological characteristic associated with the user; and
output, by the control system, the predicted at least one secondary characteristic associated with the user.

26. The non-transitory computer-readable apparatus of claim 25, wherein the predicted at least one second characteristic associated with the user relates to a location of the user, a metric associated with the location of the user, a behavioral pattern of the user, or a combination thereof.

27. The non-transitory computer-readable apparatus of claim 25, wherein the one or more sensors are configured to receive photoacoustic signals from the target object of the user.

28. The non-transitory computer-readable apparatus of claim 25, wherein:
the target object of the user comprises a blood vessel of the user; and
the at least one physiological characteristic associated with the user comprises a strain of the blood vessel, a stress of the blood vessel, a distension of the blood vessel, a stiffness of the blood vessel, a compliance of the blood vessel, a dimension of the blood vessel, or a combination thereof.

29. The non-transitory computer-readable apparatus of claim 25, wherein the at least one physiological characteristic associated with the user comprises a blood pressure of the user.

30. The non-transitory computer-readable apparatus of claim 25, wherein the predicting of the at least one secondary characteristic associated with the user comprises using one or more machine learning models implemented by the control system, the one or more machine learning models obtained by:
identifying a plurality of categories of users based on physiological characteristics determined from a training set of users and sensor signals relating to a target object of the users;
determining a correlation between the plurality of categories of users and one or more secondary characteristics associated with the users; and
generating the one or more machine learning models based on the determined correlation.
